(19) United States
(12) Patent Application Publication
(10) Pub. No.: US 2013/0011427 A1
(43) Pub. Date: Jan. 10, 2013

(54) FLAVIVIRUS SPECIES-SPECIFIC PEPTIDE TAGS FOR VACCINE AND DIAGNOSTIC USE

(75) Inventors: J. Thomas August, Baltimore, MD (US); Tin Wee Tan, Singapore (SG); Asif Mohammad Khan, Singapore (SG)

(73) Assignee: THE JOHNS HOPKINS UNIVERSITY, Baltimore, MD (US)

(21) Appl. No.: 13/516,501

(22) PCT Filed: Dec. 16, 2010
(86) PCT No.: PCT/US2010/060777

§ 371 Date: Sep. 14, 2012

Related U.S. Application Data
(60) Provisional application No. 61/287,055, filed on Dec. 16, 2009.

(51) Int. Cl. A61K 39/12; C12N 15/40; A61P 37/04; C12N 5/10; G01N 33/53; C07K 7/06; C12N 15/63

(52) U.S. Cl. 424/186.1; 530/328; 536/23.72; 435/320.1; 435/325; 435/69.1; 435/5

(57) ABSTRACT

Flaviviruses represent an increasing global public health issue, with no prophylactic and therapeutic formulations currently available for many of them. The combination of factors such as evolutionary change, global warming and wide range of animal hosts suggest the possible occurrence of Flavivirus strains with greater distribution and human pathogenicity. There is, thus, a need for greater understanding of viral protein sequences that function in the human immune responses. The evolutionary diversity of the reported sequences of major flaviviruses, such as dengue virus, yellow fever virus, Japanese encephalitis virus, and West Nile virus were analyzed with a combination of experimental and bioinformatics methodologies. The analysis of all reported sequences revealed that these species-specific peptide tags are highly conserved and are potential T-cell epitopes due to correspondence to known or predicted epitopes. These peptide tags have direct relevance to the development of new-generation vaccines and diagnostic applications.

This invention was made using funding from the U.S. government. Consequently, the U.S. government retains certain rights according to the terms of grant nos. R37A1041908 and U19 A1-056541.

### TECHNICAL FIELD OF THE INVENTION

This invention is related to the area of flaviviruses. In particular, it relates to Flavivirus species specific sequences for vaccines, constituents of vaccines, diagnostic, prophylactic, and therapeutic applications.

### BACKGROUND OF THE INVENTION

Flaviviruses, such as West Nile virus (WNV), dengue virus (DENY), Japanese encephalitis virus (JEV), and yellow fever virus (YFV), among others, are arthropod-borne RNA virus pathogens of the genus Flavivirus that have high sequence and structural homology (Kuno et al., 1998). The genome of these viruses is a positive-sense, single strand RNA, of approximately 11,000 to 12,000 nucleotides, encoding a polyprotein of approximately 3,430 amino acids that is cleaved to produce three structural proteins, capsid (C), pre-membrane (prM), membrane (M), and envelope (E), and seven non-structural (NS) proteins, NS1, 2a, 2b, 3, 4a, 4b and 5, with similar structural organization. They have become increasingly important human pathogens. For example, following the introduction of WNV in New York in 1999, the virus has become established throughout the United States as a new genotype (WN02) with multiple genetic and phenotypic changes and more efficient mosquito transmission (Davis et al., 2007; Moudy et al., 2007). Additionally, with global warming, the already widespread dengue viruses have the potential of even greater worldwide distribution. The combined problems of evolutionary change, increasing global distribution, wide range of animal as well as human hosts, and possible occurrence of strains with greater human pathogenicity, call for concerted studies with the goal of developing an effective means to combat future as well as current strains.

As many RNA viruses are pathogens in humans, there is need for increased understanding of viral protein sequences that function in the human cellular immune responses to these viruses. Several reports have described roles of CD8⁺ cytolytic T lymphocytes (CTL) and of CD4⁺ helper T lymphocytes (HTL) in the immune response to a variety of viral infections in animal model systems (BenMohamed et al., 2000; Blaney et al., 1998; Brien et al., 2007; Castrucci et al., 1994; Del Val et al., 1991; La Posta et al., 1993; Lieberman et al., 2007; Oukka et al., 1996; Purtha et al., 2007; Stemmer et al., 1999; Tsuji et al., 1998). However, knowledge of the identities and properties of both CTL and HTL immunogenic peptide sequences of pathogens is limited because of the great diversity in the recognition of the antigen peptides by the host immune system and the thousands of human leukocyte antigen molecules (HLA; human MHC) (Robinson et al., 2006); approximately 3500 reported as of June 2009 (www.ebi.ac.uk/imgt/hla/). There is also the complexity of the genetic structure of single stranded RNA viruses that are among the most variable and adaptable of subcellular parasites resulting from high frequency of point mutations during RNA replication. The genetic change, short generation times, and large virus populations result in rapid evolution dependent upon virus fitness to the vector and host. In almost all cases, the specific genetic changes responsible for viral adaptation are not known because of the stochastic nature of mutagenesis and viral fitness and the complexities of biological responses of the host. Of the great variety of T cell antigenic determinants on WNV proteins, there are only a few for which the structure is known (McMurtrey et al., 2008; Parsons et al., 2008; Wang et al., 2006). However, the advances in pathogen genome sequence data, the development of HLA transgenic mice as a model system, and large-scale synthesis of pathogen peptides has now made possible the systematic analysis of viral proteomes for protein sequences that function as T cell epitopes.

HLA Transgenic (Tg) mice are widely recognized as a leading model system for analysis of HLA-restricted T cell responses to human pathogens and disease states (Cheuk et al., 2002; Hu et al., 2005; Loirat et al., 2000; Pajot et al., 2004; Pajot et al., 2006; Pascolo, 2005; Richards et al., 2007; Sonderstrup et al., 1999; Taneja and David, 1999).

There is a continuing need in the art to identify and test Flavivirus vaccines to reduce the incidence and/or severity of Flavivirus infections and/or epidemics. The selection of evolutionarily conserved protein sequences has widely been considered important to vaccine design in order to limit the selective loss of immunity resulting from mutation and protein modification. However, sequences conserved in the evolution of viruses can be present in many different forms in viruses of related species. It is clear that exposure to multiple flaviviruses by infection or immunization will risk immune responses to a large number of cross-reactive T-cell epitopes that may, as altered peptide ligands (APL), significantly affect the immune responses to the pathogens. Memory T cells selectively engaged by a variant epitope sequence may exhibit an impaired immune response, depending on the positions and types of amino acid substitutions surrounding or within T cell epitopes and the effect of these changes on the affinity of the interaction (Ferrante and Gorski, 2007). The possible effect in humans of APL inhibition or modification of human T cell immune responses has been widely recognized (Sloan-Lancaster and Allen, 1996), particularly in relation to secondary infection and the marked cross-reactivity of memory T cells induced by primary infection followed by re-infection by a second of the four dengue serotypes (Mongkolsapaya et al., 2003; Mongkolsapaya et al., 2006; Screaton and Mongkolsapaya, 2006). The consequences of this may be relevant to the occurrence of dengue hemorrhagic fever (DHF), the more serious manifestation of the dengue virus infection (Rothman, 2004). Thus, we propose that the selection of conserved sequences that are also virus specific should have precedence in vaccine design.

### SUMMARY OF THE INVENTION

According to one aspect of the invention a polypeptide is provided. The polypeptide comprises one or more discontinuous segments of one or more proteins of a Flavivirus. The segments comprise at least 9 contiguous amino acid residues selected from SEQ ID NO: 1-206.

Another aspect of the invention is a polynucleotide which encodes a polypeptide. The polypeptide comprises one or more discontinuous segments of one or more proteins of a Flavivirus. The segments comprise at least 9 contiguous amino acid residues selected from SEQ ID NO: 1-206.

Yet another aspect of the invention is a nucleic acid vector that comprises the polynucleotide. The polynucleotide encodes the polypeptide. The polypeptide comprises one or more discontinuous segments of one or more proteins of a Flavivirus. The segments comprise at least 9 contiguous amino acid residues selected from SEQ ID NO: 1-206.

Still another aspect of the invention is a host cell. The host cell comprises a nucleic acid vector that comprises the polynucleotide. The polynucleotide encodes the polypeptide. The polypeptide comprises one or more discontinuous segments of one or more proteins of a Flavivirus. The segments comprise at least 9 contiguous amino acid residues selected from SEQ ID NO: 1-206.

According to another aspect of the invention a method is provided for producing a polypeptide. A host cell is cultured. The host cell comprises a nucleic acid vector that comprises the polynucleotide. The polynucleotide encodes the polypeptide. The polypeptide comprises one or more discontinuous segments of one or more proteins of a Flavivirus. The segments comprise at least 9 contiguous amino acid residues selected from SEQ ID NO: 1-206. The culturing is under conditions in which the host cell expresses the polypeptide.

Another aspect of the invention is a method of producing a cellular vaccine. Antigen presenting cells are transfected with a nucleic acid vector that comprises the polynucleotide. The polynucleotide encodes the polypeptide. The polypeptide comprises one or more discontinuous segments of one or more proteins of a Flavivirus. The segments comprise at least 9 contiguous amino acid residues selected from SEQ ID NO: 1-206. The antigen presenting cells thereby express the polypeptide.

An additional aspect of the invention is a method of making a vaccine. A polypeptide and an immune adjuvant are mixed together. The polypeptide comprises one or more discontinuous segments of one or more proteins of a Flavivirus. The segments comprise at least 9 contiguous amino acid residues selected from SEQ ID NO: 1-206.

A further aspect of the invention is a vaccine composition. The vaccine composition comprises a polypeptide or a polynucleotide encoding the polypeptide. The polypeptide comprises one or more discontinuous segments of one or more proteins of a Flavivirus. The segments comprise at least 9 contiguous amino acid residues selected from SEQ ID NO: 1-206.

A further aspect of the invention is a method of immunizing a human or other animal subject. A polypeptide or a nucleic acid vector or a host cell is administered to the human or other animal subject in an amount effective to elicit Flavivirus-specific T cell activation. The polypeptide comprises one or more discontinuous segments of one or more proteins of a Flavivirus. The segments each comprise at least 9 contiguous amino acid residues selected from SEQ ID NO: 1-206. The nucleic acid vector comprises a polynucleotide that encodes the polypeptide. The host cell comprises the nucleic acid vector.

Another aspect of the invention is a method of identifying a Flavivirus. A polynucleotide encoding a polypeptide comprising one or more discontinuous segments of one or more proteins of a Flavivirus or its complement is hybridized to the genome of a Flavivirus. The segments comprise at least 9 contiguous amino acid residues selected from SEQ ID NO: 1-206. Hybridization of the genome to the polynucleotide indicates a species of the Flavivirus.

Yet another aspect of the invention is a method of identifying a Flavivirus. Proteins from a virus-infected cell are contacted with an antibody which specifically binds to a polypeptide comprising one or more discontinuous segments of one or more proteins of a Flavivirus. The segments comprise at least 9 contiguous amino acid residues selected from SEQ ID NO: 1-206. Specific binding to the proteins indicates a species of Flavivirus.

Still another aspect of the invention is a method of identifying a Flavivirus. A polypeptide comprising one or more discontinuous segments of one or more proteins of a Flavivirus is contacted with a blood sample from a patient. Binding of the polypeptide to an antibody in the blood sample or T cells in the blood sample indicates a species of Flavivirus.

These and other embodiments which will be apparent to those of skill in the art upon reading the specification provide the art with new diagnostic and prophylactic reagents.

### BRIEF DESCRIPTION OF THE DRAWINGS

FIG. 1 shows a comparison of peptide and DNA immunizations.

FIG. 2 shows the number of WNV T-cell epitope peptides conserved in other flaviviruses.

FIG. 3 shows the number of flaviviruses shared by the individual WNV T-cell epitope peptides.

FIG. 4 shows the distribution of WNV epitope peptides with complete full-length occurrence in other flaviviruses.

Table 1 provides HLA-restricted T-cell epitope peptides of the WNV proteome.

Table 2 provides West Nile Virus HLA-restricted T-cell epitope peptides, class I and II. SEQ ID NOs: 211-347, in the order as shown.

Table 3A and 3B provide the apparent functional avidity of WNV T-cell epitope peptides in ELISpot assays of splenocytes from immunized HLA-transgenic mice.

Table 4 provides highly conserved WNV T-cell epitope peptides, entropy 0.1 or lower.

Table 5 provides WNV T-cell epitope peptides with high variants incidence.

Table 6 provides an example of a non-zero entropy WNV epitope peptide site. It commonly includes multiple sequences variant to the epitope, with one or more different amino acid mutations, each of which represented in a small fraction, less than 10%, of the reported sequences.

Table 7 provides WNV-specific epitope peptides.

Table 8 provides WNV T-cell epitope peptides with full-length occurrence in other flaviviruses.

Table 9 provides the distribution of cross-reactive WNV T-cell epitopes in major flaviviruses.

Table 10 provides variants of highly shared WNV epitope peptides and their incidence in other selected flaviviruses. WNV variant sequences representing less than about 10% of the corresponding database sequences were omitted.

Table 11 provides WNV HLA-restricted T-cell epitope peptides incidence and their variant incidence distribution. Entropy describing the diversity at the epitope peptide sites is also indicated.

Table 12 provides list of highly conserved and specific sequences of Flavivirus species West Nile virus (WNV), dengue virus (DENV), yellow fever virus (YFV) and Japanese encephalitis virus (JEV). SEQ ID NOs: 1-206, in the order as shown.

### DETAILED DESCRIPTION OF THE INVENTION

The inventors have identified and characterized discontinuous peptide segments from the proteomes of a number of flaviviruses. These are sequences of nine or more contiguous amino acids (aa) are highly conserved in all reported populations of the respective virus species, and are specific to the species, with no matching identity of at least nine aa in any other Flavivirus. These sequences are potential HLA-restricted epitope peptides, with many of them shown to be immunogenic in humanized HLA transgenic mice (see, e.g., Table 2 and 7) or predicted to contain T-cell epitope determinants. The identified sequences (in their nucleotide or protein form) have applications to diagnosis of virus infection and the development of new-generation vaccines. Such vaccines may be used either prophylactically or therapeutically, i.e., administered to a person who has not been infected yet or to a person who is already infected.

Discontinuous segments of the Flavivirus may be strung together to form a concatamer, if desired. They may be separated by spacer residues, optionally. Discontinuous segments are those that are not adjacent in the naturally occurring virus isolates. Segments are typically at least 9 amino acid residues and up to about 15, 16, 17, 18, 19, 20, 25, or 30 residues of contiguous amino acid residues from the virus proteome. Single segments may also be used. Because the segments are less than the whole, naturally occurring proteins, and/or because the segments are adjacent to other segments to which they are not adjacent in the proteome, the polypeptides and nucleic acids described here are non-naturally occurring.

Linkers or spacers with natural or non-naturally occurring amino acid residues may be used optionally. Particular properties may be imparted by the linkers. They may provide a particular structure or property, for example a particular kink or a particular cleavable site. Design is within the skill of the art.

Polynucleotides which encode the polypeptides may be designed and made by techniques well known in the art. The natural nucleotide sequences used by flaviviruses may be used. Alternatively non-natural nucleotide sequences may be used, including in one embodiment, human codon-optimized sequences. Design of human codon-optimized sequences is well within the skill of the ordinary artisan. Data regarding the most frequently used codons in the human genome are readily available. Optimization may be applied partially or completely.

The polynucleotides which encode the polypeptides can be replicated and/or expressed in vectors, such as DNA virus vectors, RNA virus vectors, and plasmid vectors. Preferably these will contain promoters for expressing the polypeptides in human or other mammalian or other animal cells. An example of a suitable promoter is the cytomegalovirus (CMV) promoter. Promoters may be inducible or repressible. They may be active in a tissue specific manner. They may be constitutive. They may express at high or low levels, as desired in a particular application. The vectors may be propagated in host cells for expression and collection of chimeric protein. Suitable vectors will depend on the host cells selected. In one embodiment host cells are grown in culture and the polypeptide is harvested from the cells or from the culture medium.

Suitable purification techniques can be applied to the chimeric protein as are known in the art. In another embodiment one transfects antigen presenting cells for ultimate delivery of the transfected cells to a vaccinee of a cellular vaccine which expresses and presents antigen to the vaccine. Suitable antigen presenting cells include dendritic cells, B cells, macrophages, and epithelial cells.

Polynucleotides of the invention include diagnostic DNA or RNA oligonucleotides, i.e., short sequences of proven specificity to viral species; these are sufficient to uniquely identify the viral species. Polynucleotides include oliogonucleotides such as primers and probes, which may be labeled or not. These may contain all or portions of the coding sequences for an identified conserved and specific polypeptide. Polynucleotides of the invention and/or their complements, may optionally be attached to solid supports as probes to be used diagnostically, for example, through hybridization to viral genomic sequences. Similarly, epitopic polypeptides can be attached to solid supports to be used diagnostically. These can be used to screen for activated T cells or even antibodies. Suitable solid supports include without limitation microarrays, microspheres, and microtiter wells. Antibodies may be used that are directed against the peptides as disclosed. The antibodies may be used to specifically diagnose a species of Flavivirus, for example. Polynucleotides may also be used as primers, for example, of length 18-30, 25-50, or 15-75 nucleotides, to amplify the genetic material of a specific Flavivirus. Polynucleotide primers and probes may be labeled with a fluorescent or radioactive label, if desired. These polynucleotides can be used to amplify and/or hybridize to a test sample to determine the presence or species identity of a Flavivirus. Such polynucleotides will typically be at least 18, 20, 22, 24, 26, 28, 30, 32, 34 bases in length. Any technique, including but not limited to amplification, hybridization, single nucleotide extension, and sequencing, can be used to identify the presence or species identity of a Flavivirus.

Immune adjuvants may be administered with the vaccines of the present invention, whether the vaccines are polypeptides, polynucleotides, nucleic acid vectors, or cellular vaccines. The adjuvants may be mixed with the specific vaccine substance prior to administration or may be delivered separately to the recipient, either before, during, or, after the vaccine substance is delivered. Some immune adjuvants which may be used include CpG oligodeoxynucleotides, GM-CSF, QS-21, MF-59, alum, lecithin, squalene, and Toll-like receptors (TLRs) adaptor molecules. These include the Toll-interleukin-1 receptor domain-containing adaptor-inducing beta interferon (TRIF) or myeloid differentiation factor 88 (MyD88). Vaccines may be produced in any suitable manner, including in cultured cells, in eggs, and synthetically. In addition to adjuvants, booster doses may be provided. Boosters may be the same or a complementary type of vaccine. Boosters may include a conventional live or attenuated flaviviral vaccine. Typically a high titer of antibody and/or T cell activation is desired with a minimum of adverse side effects.

Any of the conventional or esoteric modes of administration may be used, including oral, mucosal, or nasal. Additionally intramuscular, intravenous, intradermal, or subcutaneous delivery may be used. The administration efficiency may be enhanced by using electroporation. Optimization of the mode of administration for the particular vaccine composition may be desirable. The vaccines can be administered to patients who are infected already or to patients who do not yet have an infection. The vaccines can thus serve as prophylactic or therapeutic agents. One must, however, bear in mind, that no specific level of efficacy is mandated by the words prophylactic or therapeutic. Thus the agents need not be 100% effective to be vaccines. Vaccines in general are used to reduce the incidence in a population, or to reduce the risk in an individual. They are also used to stimulate an immune response to lessen the symptoms and or severity of the disease.

The above disclosure generally describes the present invention. All references disclosed herein are expressly incorporated by reference. A more complete understanding can be obtained by reference to the following specific examples, which are provided herein for purposes of illustration only, and are not intended to limit the scope of the invention.

### Example 1

We have applied this system to a large-scale analysis of HLA class I and II-restricted T cell epitopes of the WNV proteome by immunization of 6 mice transgenic for HLA proteins, 3 class 1 and 3 class II, with 452 WNV peptides covering the entire WNV proteome. WNV peptide-specific T-cell responses were assayed by IFN-γ ELISpot and the identified T cell epitope sequences were further analyzed for their apparent avidity in the ELISpot assay, conservation and diversity in the recorded WNV protein sequences, and homology to other Flavivirus pathogens. The identification and characterization of these HLA-restricted T cell epitope peptides of the WNV proteome will facilitate further analysis of the human immune response to WNV infection, including application of peptide-specific methodologies for diagnosis for virus infection and the development of new-generation vaccines.

### HLA Transgenic Mice

H-2 class II-deficient, HLA-DR2 (Vandenbark et al., 2003), HLA-DR3 (Madsen et al., 1999; Strauss et al., 1994) and -DR4/human CD4 (huCD4) (Cope et al., 1999; Fugger et al., 1994) Tg mice, and H-2 class I-deficient, HLA-A2.1 (HHD monochain) (Pascolo et al., 1997), HLA-A24/huCD8α (Lemonnier et al., unpublished) and HLA-B7 (Rohrlich et al., 2003) Tg mice were used. HLA-DR2 Tg mice express chimeric molecules, with α1 and β1 domains encoded by the HLA-DRA1*0101 and -DRB1*1501 sequences and the other domains encoded by I-Eα and I-Eβ sequences, from the I-E promoters (Vandenbark et al., 2003). HLA-DR3 Tg mice express the full-length HLA-DRA*0101 and -DRB1*0301 sequences (Madsen et al., 1999; Strauss et al., 1994). HLA-DR4/huCD4 Tg mice express the full-length HLA-DRA*0101 and -DRB1*0401 sequences from the I-Ea promoter, and the human CD4 sequence from the murine CD3δ promoter (Cope et al., 1999; Fugger et al., 1994). HLA-DR2 and -DR3 mice have a homologous deletion of the murine H-2 class II region, and HLA-DR4/huCD4 mice are deficient for I-Aβ and I-Eα. HLA-DR2 mice have a predominant C57BL/6 background, and HLA-DR3 and -DR4/huCD4 mice have mixed backgrounds (B6, B10.H2b, and DBA/1J, 129/Sv, C57BL/6, respectively). HLA-A2.1 (HHD) Tg mice express a chimeric monochain containing the HLA-A*0201α1 and α2 domains and the murine H-2D^{b} α3 domain linked to human β2-microglobulin (huβ2-m), from the HLA-A2.1 promoter, and are deficient for H-2D and murine β2-m (mβ2-m) (Pascolo et al., 1997). HLA-A24/huCD8α mice express the full-length HLA-A*2402, huβ2-m and huCD8α sequences, and are deficient for H-2K, H-2D, and mβ2-m (Lemonnier et al., unpublished). HLA-B7 mice express a chimeric heavy chain with the HLA-B*0702 α1 and α2 domains and the H-2K^{d} α3 domain, from the HLA-B7 promoter, and are deficient for H-2K and H-2D (Rohrlich et al., 2003). The three HLA class I Tg strains have been backcrossed for 6 to 12 generations on the C57BL/6 genetic background (Lemonnier, Pasteur Institute). Animals were bred and maintained at the Johns Hopkins School of Medicine Research Animal Resources facilities. Specific pathogen-free (SFP) Tg mice were derived through iodine immersion of neonates (<1 day old) and transfer to outbrcd foster females (Thompson K and Watson J, The Johns Hopkins School of Medicine). All experiments were approved by the Johns Hopkins Animal Care and Use Committee and carried out according to IACUC guidelines.

### Synthetic WNV Peptides

A library of 452 overlapping peptides covering the entire WNV proteome (NY99-flamingo 382-99 strain), each 14 to 19 amino acids in length with an overlap of 10 residues (>80% purity), was obtained as lyophilized powders from the Biodefense and Emerging Infections Research Resources Repository, NIAID, NTH (Manassas, Va.). Each was dissolved in 100% DMSO and constituted to 20% with sterile filtered water. The final concentration of each peptide was 2 μg/μl. Dissolved peptides were stored at −20° C.

### Large Scale Peptide Pool Immunization

The 6 HLA Tg mice were each immunized with the WNV peptides by use of a peptide pool protocol for large scale T cell epitope identification (Roederer and Koup, 2003). The peptides were divided into 4 immunization pools containing 1 μg of each peptide in groups of about 100 peptides each, as follows: pool 1, 88 peptides spanning the PrM/M and E proteins; pool 2, 107 peptides spanning the N, NS1, NS2a, and NS2b proteins; pool 3, 135 peptides spanning NS 3, NS4a; and NS4b proteins; pool 4, 122 peptides spanning the NS5 protein (data not shown). Each pool was mixed with 50 μl zymosan, 10 mg/ml (Sigma-Aldrich Co, St. Louis, Mo.) in PBS as adjuvant and administered subcutaneously at the base of the tail to groups of 9 to 12 mice of each Tg strain (de la Rosa et al., 2005; Goodridge et al., 2007). Initial matrix assays with peptide pools were performed on day 15-19, after one immunization. Two mice were sacrificed and their splenocytes were selectively depleted of CD8 or CD4 T cells for HLA class II and class I Tg strains, respectively (see below), and T cell responses to peptide pools (10 peptides, 1 μg peptide per pool) were assessed by IFN-γ ELISpot assays. On the following day 2 additional mice were sacrificed and the WNV peptide immunogens identified by the deconvolution analyses were individually tested by ELISpot assay. Experimental values reported herein were obtained with peptide concentration of 10 μg/ml, and a minimum of 3 assays in duplicate with different immunized mice. The remaining mice were immunized a second time on day 21 with the original peptide pool without zymosan. The mice were sacrificed on day 35 and splenocyte T cell responses to individual WNV peptides were further assessed by ELISpot assay with peptide concentrations of 10, 1 and 0.1 μg/ml. Positive control dengue virus peptides immunogenic in the relevant Tg mouse were included in each immunization protocol to evaluate the responses of the individual immunized mouse.

### DNA Immunization

The WNV strain NY99-flamingo382-99 NS3 sequence (GenBank Accession No. AF196835) with NheI and KpnI sites was optimized using the Leto 1.0 software, synthesized by Geneart Inc (Toronto, Canada), and inserted into the p43 vector (Kessler et al., 1996). A p43 vector encoding the dengue virus type 2 prM and E antigens (p-DENV-prM-E) (J. Salmon, unpublished) was used as a control. Four 6-8 weeks old female HLA-DR2 Tg mice were immunized subcutaneously at the base of the tail, twice at 3-week intervals, with 50 μg of the endotoxin-free DNA plasmid p-WNV-NS3, or p-DENV-prM-E. The mice were sacrificed on day 42 and the CD4 T cell responses to the WNV NS3 peptides were assessed by IFN-γ ELISpot assays.

### IFN-γ ELISpot Assays

Ex vivo IFN-γ ELISpot assays were performed using mouse IFN-γ ELISpot sets (BD Biosciences, San Jose, Calif.) following manufacturer's recommendations. Briefly, 96-well ELISpot plates were coated with anti-IFN-γ antibody (5 μg/ml) by incubation at 4° C. overnight, and then blocked with RPMI 1640 medium containing 10% heat-inactivated fetal calf serum, 2 mM L-glutamine, 100 μg/ml streptomycin, and 100 U penicillin, for 2 h at room temperature. Freshly isolated splenocytes from HLA class II and HLA class I Tg mice were depleted of CD8 or CD4 T cells, respectively, using magnetic beads according to the manufacturer's protocol (Miltenyi Biotech, Auburn, Calif.). Flow cytometry analysis of the depleted cells indicated this method routinely achieved >95% depletion of the targeted cells. CD8 or CD4-depleted splenocytes (100 μl containing 0.5−1.0×10⁶ cells/well) were plated together with WNV peptides. The final concentration of each peptide was 10 μg/ml in the peptide matrix pool and individual peptide validation assays, and 10, 1, and 0.1 μg/ml in the titration assays. Each peptide preparation was tested in duplicated wells. Cells plated without peptide (medium alone) served as negative controls, and concanavalin A (2.5 μg/ml; Sigma-Alrich, St. Louis, Mo.) and known HLA-restricted peptides from dengue virus serotype 3 were included as positive controls. The cells were incubated at 37° C., 5% CO₂ for 16 h. The plates were washed, incubated with biotinylated anti-IFN-γ antibody for 2 h at room temperature, followed by HRP-conjugated streptavidin for 1 h at room temperature. Detection was performed with AEC substrate (Calbiochem, San Diego, Calif.) following manufacturer's instructions. IFN-γ spot-forming cells (SFC) were counted using the Immunospot Series 3B Analyzer ELISPOT reader and Immunospot software version 3.0 (Cellular Technologies, Shaker Heights, Ohio). Experimental values were expressed as the mean numbers of SFC/10⁶ CD8- or CD4-depleted splenocytes±SD, after subtraction of values from negative controls (background). Positive ELISpot responses were defined as values above 10, and above the background plus 2 SD. Each ELISpot positive response was confirmed by three assays: matrix screening, individually by the validation assay with the individual peptide, and by peptide titration.

### WNV Sequence Data Collection and Processing for Bioinformatics Analysis

Full-length and partial WNV sequences were retrieved from the NCBI Entrez protein database (Berman et al., 2000; Wheeler et al., 2005) through the NCBI Taxonomy Browser application (taxonomy ID 11082) (as of June 2007). The sequences of the individual WNV proteins were extracted from the collected dataset by performing BLAST (Altschul et al., 1990) search against the downloaded dataset by using the individual protein sequences in the annotated WNV record P06935 as queries. Multiple sequence alignments were performed for each protein with the MUSCLE v3.6 program (Edgar, 2004) and were manually corrected for misalignments when necessary.

Entropy analysis of WNV T-cell epitope sequences

The evolutionary conservation and variability of the identified T-cell epitope regions in the recorded WNV sequences was measured by use of Shannon entropy computations (Khan et al., 2008; Shannon, 1948) in the Antigenic Variability Analyzer (AVANA) software (Miotto et al., 2008). AVANA was also used to study the representation of the individual epitope sequence and its variants in the corresponding protein alignment. At any given position x in the alignment, variant peptides were defined as those that differed by at least one amino acid from the experimentally identified T cell epitope.

T-Cell Epitope Sequence Homologies with Other Viruses

Homologs of the WNV T-cell epitopes were searched by performing BLAST analyses of all protein sequences deposited at NCBI (as of January 2009). The parameters set was as follow: limit by Entrez query “Root[ORGN] NOT txid11082[Organism:exp] NOT txid 81077[ORGN]”, “automatically adjust parameters for short sequences” option disabled, “low-complexity” filter disabled, maximum number of aligned sequences to be displayed set to “20,000”, expect threshold set to “2,000”, word size set to “2”, matrix set to “PAM30”, gap costs set to “Existence: 9, Extension: 1”, compositional adjustments set to “no adjustment”. Artificial sequence hits were removed by the “NOT txid81077[ORGN]” keyword.

### Example 2

### Identification of HLA-Restricted T Cell Epitopes of the WNV Proteome

Immunization of each of the 6 HLA transgenic mice with 4 peptide pools that comprised the 452 WNV peptides of the entire WNV proteome (data not shown), with zymosan as adjuvant, resulted in the identification of a total of 137 T cell epitope peptides, ˜30% of the 452 total, as assayed by IFN-γ ELISpot with splenocytes of the immunized mice (summarized in Table 1; complete data in Table 2). Many (43) of the 137 epitope peptides were immunogenic in multiple, 2 or more, transgenic strains, 25 of which elicited both class I and class II responses, resulting in a total of 200 individual HLA-restricted T cell responses of the 6 transgenic strains, 74 class I (40 A2, 24 A24, 10 B7) and 126 class II (50 DR2, 38 DR3, 38 DR4). These allele-specific T-cell responses to the 452 peptide immunogen thus ranged from ˜2% B7 to ˜11% DR2, with responses of 6% to 9% of the A2, A24, DR3, and DR4 mice. The M, NS3, and E proteins had the highest concentrations of immunogenic peptides; as a group they represented 160 of the 452 (35%) total peptides, but accounted for 117 (58%) of the 200 T-cell responses. The peptides of preM were non-immunogenic and the least immunogenic were peptides of NC, NS1, and NS5, which collectively elicited only 38 (19%) of the T-cell responses to 193 (43%) of the peptides. Many of the epitope peptides of M, E, and NS3 proteins were in a clustered localization (immunological hotspots). All of the M epitope peptides were in a single cluster of 17 HLA-restricted responses, E contained 3 clusters of the protein amino acids 39-85, 119-152, and 426-482, and almost all of the NS3 peptide epitopes were in clusters of amino acids 1-115, 138-282, 304-376, and 455-605. These 8 clustered regions collectively comprise 65, almost 50%, of the 137 epitope peptide sequences.

TABLE 1
HLA-restricted T-cell epitope peptides of the WNV proteome.

| | | Protein | ELISpot | | | | | | | |
|---|---|---|---|---|---|---|---|---|---|---|
| | Protein | peptides | positive | | | | | | | |
| WNV | size | analyzed | peptides | HLA-restricted T cell activation | | | | | | |
| protein | (aa)^{a} | (#) | # (%) | A2 | A24 | B7 | DR2 | DR3 | DR4 | Total |
| C | 123 | 15 | 1 (~7%) | 0 | 0 | 1 | 0 | 0 | 0 | 1 |
| prM | 167 | 21 | 6 (~29%) | 1 | 2 | 1 | 6 | 3 | 4 | 17 |
| E | 501 | 67 | 25 (~37%) | 8 | 6 | 2 | 8 | 12 | 6 | 42 |
| NS1 | 352 | 46 | 10 (~22%) | 2 | 1 | 0 | 1 | 3 | 5 | 12 |
| NS2a | 231 | 30 | 10 (~33%) | 0 | 3 | 0 | 8 | 3 | 3 | 17 |
| NS2b | 131 | 16 | 6 (~38%) | 1 | 3 | 1 | 2 | 0 | 2 | 9 |
| NS3 | 619 | 84 | 46 (~55%) | 26 | 0 | 1 | 16 | 11 | 4 | 58 |
| NS4a | 149 | 18 | 5 (~28%) | 0 | 3 | 0 | 1 | 1 | 2 | 7 |
| NS4b | 255 | 33 | 9 (~27%) | 0 | 1 | 0 | 3 | 3 | 5 | 12 |
| NS5 | 905 | 122 | 19 (~16%) | 2 | 5 | 4 | 5 | 2 | 7 | 25 |
| Total | 3433 | 452 | 137 (~30%) | 40 | 24 | 10 | 50 | 38 | 38 | 200 |

| | | | | | | |
|---|---|---|---|---|---|---|
| Frequency allele-specific epitopes per total | 9% | 5% | 2% | 11% | 8% | 8% |
| proteome peptides | | | | | | |

^{a}Size indicated in number of amino acids with respect to the flamingo strain (NCBI accession no. AAF20092.2).

TABLE 2
West Nile virus HLA-restricted T-cell epitope peptides, class I and II. (SEQ ID NOs: 211-347)

| ELISpot positive peptides | | T cell activation of HLA transgenic mice | | | | | |
|---|---|---|---|---|---|---|---|
| Protein & | | (SFC/10⁶) IFN-γ ELISpot | | | | | |
| position^{a} | Peptide sequence^{b,c} | A2 | A24 | B7 | DR2 | DR3 | DR4 |
| C 9-25 | GKSRAVN⁴M¹LKRGMPRVL | | | 43 ± 17 | | | |
| prM 114-130 | STKATR²Y¹L⁴V⁵KTESWILR | | | | 101 ± 33 | | |
| prM 121-138 | L⁴VKTES⁴WI³LRNPGYALVA | | | | 130 ± 23 | | 56 ± 6 |
| prM 129-145 | LRN³PG²YALVAAVIGWML | | 71 ± 38 | 31 ± 7 | 62 ± 11 | | |
| prM 136-153 | LVAAVI⁴GW⁶M^{5,6}LGSNTMQRV | | | | 235 ± 9 | 78 ± 35 | 327 ± 162 |
| prM 144-161 | M^{5,6}LGSN¹T¹MQRV¹VFVVLLLL | | 59 ± 18 | | 84 ± 32 | 134 ± 35 | 315 ± 157 |
| prM 152-167 | RV¹VF^{4,6}VV¹LLLLVAPAYS | 318 ± 179 | | | 269 ± 30 | 90 ± 47 | 117 ± 10 |
| E 39-56 | P⁵TID⁶VKM¹M⁶NMEAANLAEV | | 137 ± 124 | | | | |
| E 54-71 | AEVR¹SY⁶CY⁶LATVSDLSTK | | | | | | 205 ± 56 |
| E 62-77 | LATVSDLSTKAACPTM | 465 ± 343 | | | | | 47 ± 4 |
| E 68-85 | LSTKAACPTMGEAHNDKR | 372 ± 242 | | | | | |
| E 99-113 | RGWGNGCGLFGKGSI | | | | | 79 ± 7 | |
| E 119-136 | FACSTKAIGRTILKENIK | | 179 ± 136 | | | | |
| E 127-144 | GRTILKENI^{4,6}KYEVAIFVH | | | | 120 ± 1 | 198 ± 21 | |
| E 135-152 | I^{4,6}KYEVAIF⁶VHGPTTVESH | | | | | 86 ± 13 | |
| E 171-188 | PAAPSYTLKLGEYGEVTV | 279 ± 217 | 533 ± 333 | 996 ± 100 | | | |
| E 195-212 | G¹IDTNAYY⁶VMTVGTKTFL | | 137 ± 70 | | | | 53 ± 16 |
| E 210-225 | TF¹LVHREW^{5,6}FMDLNLPW | 323 ± 175 | 454 ± 412 | 519 ± 477 | | | |
| E 238-255 | TLMEFEE³PHATKQSVIAL | | 122 ± 108 | | | | |
| E 293-310 | EKLQ¹LKGTT²YGVCSKAFK | | | | | | 47 ± 5 |
| E 356-373 | VTV⁴NPF⁶VS¹VATANAKVLI | | | | | | 217 ± 51 |
| E 370-386 | KVLIELEPPFGDSYIVV | | | | | | 148 ± 10 |
| E 384-399 | IVVGRGEQQINHHWHK | | | | | 87 ± 16 | |
| E 390-407 | EQQINHHWHKSGSSIGKA | | | | | 50 ± 6 | |
| E 398-415 | HKSGSSIGKAFTTTLKGA | | | | 168 ± 18 | 88 ± 1 | |
| E 426-443 | WDFGS¹VG⁴GVFTSVGKAVH | | | | | 83 ± 13 | |
| E 434-451 | VFTSVGKAVHQVFGGAFR | | | | 116 ± 5 | 79 ± 19 | |
| E 442-459 | VHQVF⁵G⁴GAF⁶RSLFGGMSW | 442 ± 313 | | | 117 ± 5 | 101 ± 15 | |
| E 450-467 | FRS¹L⁴FGG¹MSWITQGLLGA | 205 ± 145 | | | 261 ± 13 | 104 ± 21 | |
| E 458-474 | SWITQGL¹LGALLLWMGI | | | | 51 ± 4 | | |
| E 465-482 | LGAL^{1,4}L⁶LWMGINARDRSIA | 310 ± 186 | | | 490 ± 64 | 330 ± 91 | |
| E 486-501 | LA¹VGGV^{1,4}L⁶L⁶FLSVNVHA | 323 ± 173 | | | 214 ± 27 | 244 ± 16 | |
| NS1 1-19 | DTGCAI⁵DISRQELRCGSGV | | | | | 572 ± 20 | |
| NS1 10-27 | RQELRCGSGVFIHNDVEA | | | | | | 63 ± 8 |
| NS1 18-35 | GVFIHNDVEAWMDRYKYY | | | | | 52 ± 8 | |
| NS1 161-178 | GLTSTRMFL⁶KVRESNTTE | | | | 134 ± 8 | | |
| NS1 205-221 | RLNDTW^{4,6}KLERAVLGEVK | | | | | | 61 ± 15 |
| NS1 228-245 | THTLWGDGILESDLIIPV | 84 ± 47 | | | | | |
| NS1 236-251 | ILESDL⁶IIPVTLAGPR | 78 ± 42 | 43 ± 18 | | | | |
| NS1 270-287 | EGRVEIDFDYCPGTTVTL | | | | | | 54 ± 6 |
| NS1 305-322 | GKL⁵ITDWCCRSCTLPPLR | | | | | 557 ± 25 | 109 ± 22 |
| NS1 327-343 | SGCWY⁵GMEIRPQRHDEK | | | | | | 78 ± 4 |
| NS2a 6-23 | IDPFQ¹LGLLV¹VFLATQEV | | 437 ± 62 | | 563 ± 41 | 45 ± 7 | 234 ± 8 |
| NS2a 45-61 | VFGGI⁵TYTDVLRYVILV | | 243 ± 196 | | 307 ± 119 | | 74 ± 11 |
| NS2a 52-66 | TDV⁴LRY^{3,6}VILVGAAFA | | | | 516 ± 9 | | |
| NS2a 65-81 | FAESNSGGDVVHLALMA | | | | | | 60 ± 13 |
| NS2a 80-97 | MATF⁶KIQ³PV⁴F¹MVASFLKA | | | | 289 ± 83 | | |
| NS2a 128-145 | EIPDVLNS¹LAVAWMILRA | | | | 384 ± 125 | | |
| NS2a 136-154 | LAVAW⁴MI⁶LRAI¹TFTTTSNV | | | | 98 ± 22 | | |
| NS2a 160-177 | ALLTPGLRC¹L⁵NLDVYRIL | 63 ± 18 | | | | 59 ± 3 | |
| NS2a 168-183 | C¹LN¹LDV⁴YRILLLMVGI | | | | 556 ± 33 | 231 ± 16 | |
| NS2a 211-229 | G¹LFN³PMILAAGLIACDPNR | | | | 74 ± 23 | | |
| NS2b 1-18 | GWPATEVM¹TAVGLMFAIV | | | | | | 104 ± 18 |
| NS2b 39-56 | MFAAFVISGKSTDMWIER | | | | 47 ± 18 | | |
| NS2b 59-75 | DISWESDAEITGSSERV | | | | 59 ± 12 | | |
| NS2b 84-101 | NF^{1,6}QL⁵MNDPGAPWKIWMLR | | 35 ± 1 | | | | |
| NS2b 107-124 | ISAYTPW^{4,6}AILPSVVGFWI | | 50 ± 11 | 81 ± 8 | | | 163 ± 4 |
| NS2b 115-131 | ILPS¹VVGFWITLQYTKR | 108 ± 60 | 56 ± 2 | | | | |
| NS3 1-15 | GGV⁵LWDTPSPKEYKK | | | 45 ± 12 | | | |
| NS3 6-23 | DTPSPKEYKKGDTTTGVY | | | | | | 43 ± 6 |
| NS3 22-38 | V⁴YRIMTRGL¹LGSYQAGA | | | | 155 ± 22 | | |
| NS3 37-54 | GAGV¹MVEGVFHTLWHTTK | | | | 149 ± 13 | 368 ± 66 | |
| NS3 45-59 | VFHTLW⁶HTTKGAALM | | | | | 63 ± 10 | |
| NS3 50-65 | WHTTKGAA¹LMSGEGRL | | | | | 191 ± 37 | |
| NS3 70-87 | GSVKEDRL⁴CYGGPWKLQH | 174 ± 13 | | | | | |
| NS3 78-95 | CYGGPWKLQHKWNGQDEV | 150 ± 17 | | | | | |
| NS3 85-100 | LQHKWNG¹QDEVQMIVV | 72 ± 9 | | | | | |
| NS3 91-107 | G¹QDEV⁶QMIVVEPGKNVK | 156 ± 11 | | | | | |
| NS3 98-115 | IVVEPGKNVKNVQTKPGV | 145 ± 25 | | | | | |
| NS3 138-155 | PIVDKNGDV⁴IGLYGNGVI | 159 ± 10 | | | | | |
| NS3 146-162 | V⁴I⁴GLY⁶GNGVIMPNGSYI | 58 ± 38 | | | | | |
| NS3 161-177 | YISAIVQGERMDEPIPA | 56 ± 28 | | | 72 ± 15 | | |
| NS3 176-192 | PAGFEPEM¹LRKKQITVL | | | | 59 ± 8 | | |
| NS3 183-200 | M¹LRKKQITVLDLHPGAGK | 62 ± 24 | | | 55 ± 2 | | |
| NS3 199-216 | GKTRRIL³PQIIKEAINRR | | | | 52 ± 6 | | |
| NS3 206-223 | PQIIKEAIN⁴RRLRTAVLA | 108 ± 22 | | | 323 ± 21 | | |
| NS3 214-230 | NRRL⁶R⁴TA³VLAPTRVVAA | | | | 146 ± 29 | | |
| NS3 221-237 | VLAPTRVVAAEMAEALR | 26 ± 4 | | | | | |
| NS3 228-243 | VAAEMA⁴EALRGLPIRY | | | | | | 46 ± 9 |
| NS3 241-258 | IRY⁶QTSAVPREHNGNEIV | 127 ± 18 | | | | | |
| NS3 249-266 | PREHNGNEIVDVMCHATL | 79 ± 27 | | | 139 ± 25 | | 46 ± 16 |
| NS3 265-282 | TLT⁴HRLMSPHRVPNYNLF | 150 ± 30 | | | 169 ± 16 | | |
| NS3 304-321 | KVELGEAAAIFMTATPPG | 94 ± 22 | | | | | |
| NS3 317-333 | AT³PPGTSDPFPESNSPI | 151 ± 18 | | | | | |
| NS3 324-340 | DPFPESNSPISDLQTEI | 117 ± 20 | | | | | |
| NS3 336-352 | LQTEIPDRAWNSGYEWI | 167 ± 35 | | | | | |
| NS3 343-360 | RAWNSGYEWITEYTGKTV | 55 ± 21 | | | | | |
| NS3 359-376 | TVWF^{5,6}VPSVKIAGNEIALCL | 75 ± 44 | | | | | |
| NS3 413-430 | EM⁵G⁴ANF⁶KASR³VIDSRKSV | 130 ± 15 | | | | | |
| NS3 455-469 | AAQRRGRIGRNPSQV | | | | 200 ± 27 | | |
| NS3 460-475 | GRIGRNPSQVGDEYCY | | | | 123 ± 4 | | |
| NS3 474-489 | CYGGHTNEDDSNFAHW | 92 ± 18 | | | | 83 ± 23 | |
| NS3 480-496 | NEDDSNFAHWTEARIML | | | | | 121 ± 1 | |
| NS3 487-501 | AHWTEARIMLDNINM | 308 ± 188 | | | | 431 ± 112 | |
| NS3 492-509 | ARIM⁶LDNINM³PNGLIAQF | | | | | 216 ± 42 | |
| NS3 500-517 | NMPNGLIAQFYQPEREKV | | | | 51 ± 10 | 145 ± 49 | |
| NS3 515-532 | EKVY¹TMDGEYRLRGEERK | 140 ± 24 | | | 95 ± 27 | | |
| NS3 523-539 | EYRLRGEERKNFLELLR | 57 ± 11 | | | 160 ± 48 | | |
| NS3 530-547 | ERKNFL⁴ELLR¹TADLPVWL | 127 ± 10 | | | | | |
| NS3 545-562 | VW¹LAYKVAAAGVSYHDRR | | | | | 110 ± 9 | |
| NS3 560-574 | DRRWCF⁶DGPRTNTIL | | | | | 225 ± 10 | |
| NS3 565-582 | FDGPRTNT¹ILEDNNEVEV | | | | | 59 ± 3 | |
| NS3 581-597 | EVITK⁵LGERKILRPRWI | | | | | | 52 ± 11 |
| NS3 588-605 | ERKILR³PRWIDARVYSDH | | | | 42 ± 4 | | |
| NS4a 1-17 | S¹QIG¹LIEVLGKMPEHFM | | | | | 132 ± 17 | |
| NS4a 15-31 | HF¹MGK¹TWEA¹LDTMYVVA | | | | 278 ± 26 | | 187 ± 20 |
| NS4a 54-71 | I¹A¹L⁴IAL¹LSV¹M^{1,2}TMGVFFLL | | 70 ± 16 | | | | |
| NS4a 62-79 | V¹M^{1,2}TMGVFFLLMQRKGIGK | | 117 ± 39 | | | | |
| NS4a 86-103 | VLGVAT¹FFCWMAEVPGTK | | 65 ± 30 | | | | 63 ± 1 |
| NS4b 30-47 | GEF^{1,5,6}LLDL⁴R³PA¹TAWSLYAV | | | | 89 ± 21 | | |
| NS4b 38-55 | PA¹TAWS¹L^{2,4}Y⁶AVTTAVLTPL | | | | 57 ± 11 | 67 ± 13 | |
| NS4b 63-80 | DY¹I⁶N⁴TS¹L⁶TSINVQASALF | | | | | 386 ± 60 | 92 ± 13 |
| NS4b 87-103 | PF¹VDVGVSALLLAAGCW | | | | | | 59 ± 14 |
| NS4b 101-118 | GCWG¹QV⁶TLTVTVTAATLL | | | | | | 134 ± 8 |
| NS4b 187-204 | VV⁵NPSVKTVREAGILITA | | | | | | 83 ± 13 |
| NS4b 201-218 | LITAAAV⁴TLWENGASSVW | | | | | 99 ± 26 | 73 ± 8 |
| NS4b 233-250 | GWLS¹CL⁶SITW^{4,6}TLIKNMEK | | | | 62 ± 6 | | |
| NS4b 241-255 | TW^{4,6}TLIKNMEKPGLKR | | 53 ± 32 | | | | |
| NS5 115-132 | L¹V⁴QS²YGWNI⁴VTMKSGVDV | | | | | 79 ± 23 | |
| NS5 145-162 | CDIGESSSSAEVEEHRTI | | | 50 ± 22 | | | |
| NS5 153-168 | SAEV⁴EEHRTIRVLEMV | 332 ± 52 | | 112 ± 72 | 250 ± 24 | | |
| NS5 181-198 | V⁶KV¹LCPY¹MPK¹VIEKMELL | | | | 122 ± 40 | | |
| NS5 212-229 | SRNSTHEMYWVSRASGNV | | | | 168 ± 13 | | |
| NS5 448-463 | ECHTCIYNMMGKREKK | 26 ± 7 | | | | | |
| NS5 470-486 | AKGSRA^{1,4}IW²F^{1,4}MWLGARFL | | 183 ± 19 | | | | |
| NS5 477-494 | W²F^{1,4}MWLGARFLEFEALGFL | | 198 ± 60 | | | | |
| NS5 598-615 | REDQRGSGQVVTYALNTF | | | | 252 ± 41 | | |
| NS5 605-622 | GQVV⁶TY¹A^{1,6}L^{4,6}NTFTNLAVQL | | | | 556 ± 27 | | 240 ± 76 |
| NS5 613-631 | NTF¹TNLAVQLVRMMEGEGV | | | | | | 119 ± 21 |
| NS5 704-721 | GWYDWQQVPFCSNHFTEL | | | | | | 65 ± 27 |
| NS5 755-772 | DTACLAK¹S²Y⁶AQMWLLLYF | | 132 ± 62 | | | | |
| NS5 763-780 | Y⁶AQMWLLL⁵YFHRRDLRLM | | | 36 ± 16 | | | 91 ± 32 |
| NS5 771-788 | YFH5RRDL4,6RL¹MANAICSAV | | | | | | 135 ± 47 |
| NS5 791-808 | NWVPTGRTTWSIHAGGEW | | | | | | 78 ± 17 |
| NS5 828-843 | WMEDKTPVEKWSDVPY | | | 39 ± 14 | | | |
| NS5 842-859 | PYSGKREDIWCGSLIGTR | | 180 ± 66 | | | | |
| NS5 863-879 | TWAENI^{4,5,6}QVAINQVRAII | | 79 ± 11 | | | 121 ± 2 | 33 ± 1 |

^{a}Sequence positions in boldface are those with more than one FILA-restricted T-cell response.
^{b}Underlined amino acids represent overlapping sequences of adjacent epitope peptides.
^{c}The amino acid residues with the superscript numbers 1 to 6 refer to the first residue of the HLA class I nonamer binders (1: A*0201; 2: A*2402; and 3: B*0702) or the nonamer core of the HLA class II binders (4: DRB1*1501; 5: DRB1*0301; and 6: DRB1*0401) predicted by the NetMHC 3.2 [48] (www.cbs.dtu.dk/services//NetMHC-3.2/) and NetMHCIIPan 1.0 [49] (www.cbs.dtu.dk/services/NetMHClIpan/) Web-server immunionformatic algorithims. Those sequences without a superscript number 1 to 6 did not have a predicted binder.

As further characterization of the epitope peptides, their avidity in the IFN-γ ELISpot assay, which can be attributed either to the binding reaction of either the HLA or T-cell receptor molecules, was measured by titration of the peptides over a 100-fold range of concentrations (10, 1, and 0.1 μg/ml). The majority of the peptide epitopes, 37 of 74 HLA-restricted class 1 responses and 83 of 126 class II, demonstrated high functional avidity (Table 3). Notably, while 10 μg/ml resulted in the greatest number (200) of responses, only 54 required the high peptide concentration (10 μg) to elicit T-cell activation following 2 immunizations with the peptide pools. A majority of the assays of the combined class I and class II T-cell responses (120 of 200) were positive at 0.1 μg/ml peptide, including all M and E class II T-cell responses and peptides of each protein except for class I NS3 and NS5, and class II NS3 and NS4b responses. Many of the peptides with high T-cell response scores (>200 SFC per 1×106 splenocytes) demonstrated comparable T-cell responses in assays with 1.0 and 0.1 μg/ml peptide.

TABLE 3A AND 3B
The apparent functional avidity of WNV T-cell epitope peptides in ELISpot assays of
splenocytes from immunized HLA-transgenic mice.

| WNV | T-cell responses (#)^{a} | | | | | | | | | | | | | | | |
|---|---|---|---|---|---|---|---|---|---|---|---|---|---|---|---|---|
| protein | L | I | H | Total | L | I | H | Total | L | I | H | Total | L | I | H | Total |

3A. HLA class I-restricted T-cell responses

| | A2 | | | | A24 | | | | B7 | | | | A2, A24, B7 | | | |
|---|---|---|---|---|---|---|---|---|---|---|---|---|---|---|---|---|
| C | 0 | 0 | 0 | 0 | 0 | 0 | 0 | 0 | 0 | 0 | 1 | 1 | 0 | 0 | 1 | 1 |
| prM | 0 | 0 | 1 | 1 | 0 | 0 | 2 | 2 | 1 | 0 | 0 | 1 | 1 | 0 | 3 | 4 |
| E | 2 | 2 | 4 | 8 | 0 | 0 | 6 | 6 | 2 | 0 | 0 | 2 | 4 | 2 | 10 | 16 |
| NS1 | 1 | 0 | 1 | 2 | 0 | 0 | 1 | 1 | 0 | 0 | 0 | 0 | 1 | 0 | 2 | 3 |
| NS2a | 0 | 0 | 0 | 0 | 0 | 0 | 3 | 3 | 0 | 0 | 0 | 0 | 0 | 0 | 3 | 3 |
| NS2b | 0 | 0 | 1 | 1 | 1 | 0 | 2 | 3 | 0 | 1 | 0 | 1 | 1 | 1 | 3 | 5 |
| NS3 | 7 | 9 | 10 | 26 | 0 | 0 | 0 | 0 | 1 | 0 | 0 | 1 | 8 | 9 | 10 | 27 |
| NS4a | 0 | 0 | 0 | 0 | 1 | 1 | 1 | 3 | 0 | 0 | 0 | 0 | 1 | 1 | 1 | 3 |
| NS4b | 0 | 0 | 0 | 0 | 1 | 0 | 0 | 1 | 0 | 0 | 0 | 0 | 1 | 0 | 0 | 1 |
| NS5 | 1 | 0 | 1 | 2 | 1 | 1 | 3 | 5 | 2 | 2 | 0 | 4 | 4 | 3 | 4 | 11 |
| Total | 11 | 11 | 18 | 40 | 4 | 2 | 18 | 24 | 6 | 3 | 1 | 10 | 21 | 16 | 37 | 74 |

3B. HLA class II-restricted T-cell responses

| | DR-2 | | | | DR-3 | | | | DR-4 | | | | DR-2, -3, -4 | | | |
|---|---|---|---|---|---|---|---|---|---|---|---|---|---|---|---|---|
| C | 0 | 0 | 0 | 0 | 0 | 0 | 0 | 0 | 0 | 0 | 0 | 0 | 0 | 0 | 0 | 0 |
| prM | 0 | 0 | 6 | 6 | 0 | 0 | 3 | 3 | 0 | 0 | 4 | 4 | 0 | 0 | 13 | 13 |
| E | 0 | 0 | 8 | 8 | 0 | 0 | 12 | 12 | 0 | 0 | 6 | 6 | 0 | 0 | 26 | 26 |
| NS1 | 0 | 0 | 1 | 1 | 0 | 0 | 3 | 3 | 2 | 0 | 3 | 5 | 2 | 0 | 7 | 9 |
| NS2a | 0 | 0 | 8 | 8 | 2 | 1 | 0 | 3 | 1 | 0 | 2 | 3 | 3 | 1 | 10 | 14 |
| NS2b | 0 | 0 | 2 | 2 | 0 | 0 | 0 | 0 | 1 | 0 | 1 | 2 | 1 | 0 | 3 | 4 |
| NS3 | 7 | 3 | 6 | 16 | 6 | 2 | 3 | 11 | 3 | 0 | 1 | 4 | 16 | 5 | 10 | 31 |
| NS4a | 0 | 0 | 1 | 1 | 1 | 0 | 0 | 1 | 1 | 0 | 1 | 2 | 2 | 0 | 2 | 4 |
| NS4b | 2 | 0 | 1 | 3 | 3 | 0 | 0 | 3 | 1 | 1 | 3 | 5 | 6 | 1 | 4 | 11 |
| NS5 | 1 | 2 | 2 | 5 | 0 | 0 | 2 | 2 | 2 | 1 | 4 | 7 | 3 | 3 | 8 | 14 |
| Total | 10 | 5 | 35 | 50 | 12 | 3 | 23 | 38 | 11 | 2 | 25 | 38 | 33 | 10 | 83 | 126 |

^{a}Low (L) avidity T-cell determinants defined as those IFN-γ ELISpot positive only at 10 μg/ml; intermediate (I), 10 and 1 μg/ml; and high (H), 10, 1 and 0.1 μg/ml.

### Example 3

### Correspondence Between Peptide and DNA Plasmid Immunization

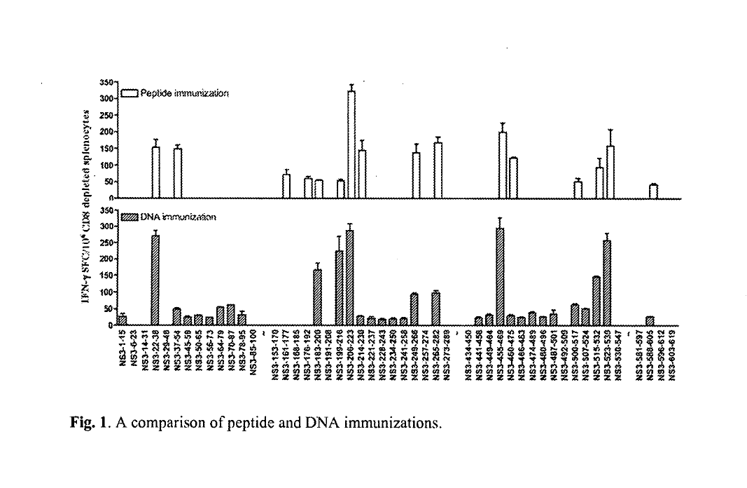

T-cell epitopes elicited by peptide immunization with adjuvant may differ from those elicited by viral infection because of the many differences in antigen delivery and processing, and the mechanisms involved in activation of the cellular immune response system. As an evaluation of the extent of these possible differences, the T-cell responses of DR2 transgenic mice immunized with NS3 peptides were compared to the responses to a DNA plasmid immunogen encoding the NS3 protein. The DNA construct was designed to encode NS3 as a cytoplasmic protein lacking a signal sequence and a transmembrane domain and therefore possibly subject to a processing pathway comparable to that of the NS3 proteolytically released from the viral proteome polyprotein. All but two of the peptide-specific T-cell responses following peptide immunization were also detected after DNA immunization (FIG. 1). The correspondence between the two immunizations was greatest with the stronger T-cell responses, especially those of >100 SFC/10⁶ CD8 depleted splenocytes. The major difference was a greater number of low T-cell responses (<50 SFC/10⁶ CD8 depleted splenocytes) with DNA immunization. The data suggest that while processing pathways can be important in epitope activation of T cells, there are common mechanisms for selection of epitope peptides delivered as extracellular peptides or encoded by DNA for cellular synthesis.

### Example 4

### Evolutionary Conservation and Diversity of the WNV T-Cell Epitope Peptides

Analysis of the evolutionary conservation and the variability of the T-cell epitope peptides identified in this study was performed to determine the distribution of these sequences in the known sequence dataset of WNV. A total of 2,746 complete and partial WNV protein sequences were extracted from the NCBI Entrez protein database (as of June 2007): C, 264; prM, 417; E, 927; NS1, 164; NS2a, 143; NS2b, 146; NS3, 146; NS4a, 142; NS4b, 141; and NS5, 256. The majority of WNV T-cell epitope peptides were highly conserved. All but 12 had an entropy of less than 1.0 (average 0.48) (Table 11) and 31 peptides with entropies of 0.1 or less, mainly present in the E, NS3, and NS5 proteins were found as the unmodified sequence in 99% or more of all WNV (Table 4). There were only 7 epitope peptides of the E, NS2a, NS3, NS4A, and NS5 proteins with entropies greater than 0.7 that included variant sequences present in more than 10% (11 to 23%) of the recorded WNV protein sequences (Table 5). However, it is noteworthy that each peptide epitope, except for sequences with entropy of 0.0 (completely conserved epitopes), were represented by multiple variant sequences with one or more amino acid mutations in a small fraction, less than 10%, of the reported sequences, and in many cases, less than 1%. For example, envelope peptide from position 62 to 77 aa with an entropy of 0.5, was present in 94.3% of the database sequences, while the remaining 5.7% of the database sequences were represented by 16 different variants, 14 of which were each present in less than 1.0% of the recorded viral sequences (Table 6). The origin of these apparently rare sequences is uncertain. Possibly, they represent an under-sequenced clade that is common in nature but localized to a region where the virus was not widely studied.

TABLE 4
Highly conserved WNV T-cell epitope peptides,
entropy 0.1 or lower.

| Protein & | | Incidence | |
|---|---|---|---|
| position | Peptide sequence | (%)^{a} | Entropy |
| prM 114-130 | STKATRYLVKTESWILR | >99 | 0.1 |
| prM 121-138 | LVKTESWILRNPGYALVA | >99 | 0.1 |
| E 99-113 | RGWGNGCGLFGKGSI | >99 | 0.1 |
| E 135-152 | IKYEVAIFVHGPTTVESH | 99 | 0.1 |
| E 293-310 | EKLQLKGTTYGVCSKAFK | 99 | 0.1 |
| E 370-386 | KVLIELEPPFGDSYIVV | >99 | 0.0 |
| E 384-399 | IVVGRGEQQINHHWHK | >99 | 0.1 |
| E 390-407 | EQQINHHWHKSGSSIGKA | 99 | 0.1 |
| E 450-467 | FRSLFGGMSWITQGLLGA | >99 | 0.1 |
| E 458-474 | SWITQGLLGALLLWMGI | >99 | 0.1 |
| E 465-482 | LGALLLWMGINARDRSIA | 99 | 0.1 |
| NS1 305-322 | GKLITDWCCRSCTLPPLR | 99 | 0.1 |
| NS2b 1-18 | GWPATEVMTAVGLMFAIV | >99 | 0.1 |
| NS3 45-59 | VFHTLWHTTKGAALM | 99 | 0.1 |
| NS3 50-65 | WHTTKGAALMSGEGRL | 99 | 0.1 |
| NS3 138-155 | PIVDKNGDVIGLYGNGVI | 99 | 0.1 |
| NS3 183-200 | MLRKKQITVLDLHPGAGK | 99 | 0.1 |
| NS3 265-282 | TLTHRLMSPHRVPNYNLF | 100 | 0.0 |
| NS3 359-376 | TVWFVPSVKMGNEIALCL | 99 | 0.1 |
| NS3 487-501 | AHWTEARIMLDNINM | >99 | 0.1 |
| NS3 565-582 | FDGPRTNTILEDNNEVEV | 99 | 0.1 |
| NS5 115-132 | LVQSYGWNIVTMKSGVDV | >99 | 0.1 |
| NS5 470-486 | AKGSRAIWFMWLGARFL | >99 | 0.1 |
| NS5 477-494 | WFMWLGARFLEFEALGFL | 100 | 0.0 |
| NS5 598-615 | REDQRGSGQVVTYALNTF | 100 | 0.0 |
| NS5 605-622 | GQVVTYALNTFTNLAVQL | 99 | 0.1 |
| NS5 613-631 | NTFTNLAVQLVRMMEGEGV | 99 | 0.1 |
| NS5 704-721 | GWYDWQQVPFCSNHFTEL | 100 | 0.0 |
| NS5 755-772 | DTACLAKSYAQMWLLLYF | >99 | 0.1 |
| NS5 763-780 | YAQMWLLLYFHRRDLRLM | >99 | 0.1 |
| NS5 771-788 | YFHRRDLRLMANAICSAV | 100 | 0.0 |
| NS5 842-859 | PYSGKREDIWCGSLIGTR | 99 | 0.1 |

^{a}Percentage incidence (rounded off to the nearest whole number) of the epitope peptide sequence in all reported WNV sequences. Those with >99% incidence do not include 100%. SEQ ID NOs for each peptide are identified in Table 2.

TABLE 5
WNV T-cell epitope peptides with
high variants incidence.

| Protein & | | Incidence | En- |
|---|---|---|---|
| position^{a} | Peptide sequence^{b} | (%)^{c} | tropy |
| E 119-136 | FACSTKAIGRTILKENIK | 81 | 0.9 |
| | .......T.......... | 16 | |
| | ...T...T.WI.Q..... | 2 | |
| | 11 variants | <1 each | |
| NS2a 168-183 | CLNLDVYRILLLMVGI | 78 | 1.0 |
| | ...............V | 18 | |
| | .........V...I.. | 1 | |
| | 4 variants | <1 each | |
| NS2a 211-229 | GLFNPMILAAGLIACDPNR | 75 | 1.3 |
| | .............T..... | 12 | |
| | .V..........M...... | 7 | |
| | .F..........V...... | 3 | |
| | .M.S.LV............ | 1 | |
| | 3 variants | <1 each | |
| NS3 317-333 | ATPPGTSDPFPESNSPI | 87 | 0.7 |
| | ..............A.. | 11 | |
| | 4 variants | <1 each | |
| NS3 343-360 | RAWNSGYEWITEYTGKTV | 69 | 1.3 |
| | .............I.... | 23 | |
| | ....T........V.... | 6 | |
| | .............V.... | 1 | |
| | 1 variant | <1 | |
| NS4a 54-71 | IALIALLSVMTMGVFFLL | 80 | 1.1 |
| | ....T............. | 11 | |
| | ..........SL...... | 4 | |
| | .V........SL...... | 3 | |
| | 4 variants | <1 each | |
| NS5 863-879 | TWAENIQVAINQVRAII | 71 | 1.6 |
| | ..............S.. | 12 | |
| | ......H.......SV. | 9 | |
| | ....D.........S.. | 2 | |
| | ......H.......SL. | 2 | |
| | 10 variants | <1 each | |

^{a}Epitope peptide sites (7), each with entropy greater than 0.7, that included at least one variant that was present in more than 10% of the recorded WNV protein sequences.
^{b}Evolutionary variants of the WNV predominant epitope peptide sequences (in bold face) are indicated with only the variant amino acids. Variant(s) with less than 1% incidence are indicated by the sum of the number of such variants.
^{c}Percentage incidence (rounded off to the nearest whole number) of the epitope peptide sequence and its variants in all WNV sequences analyzed. Those with <1% incidence do not include 0%. SEQ ID NOs for each peptide are identified in Table 2.

TABLE 6
An example of a non-zero entropy WNV epitope
peptide site. It commonly includes multiple
sequences variant to the epitope, with one or
more different amino acid mutations, each of
which represented in a small fraction,
less than 10%, of the reported sequences.

| Protein & | | Incidence | |
|---|---|---|---|
| position | Peptide sequence^{a} | (%)^{b} | Entropy |
| E 62-77 | LATVSDLSTKAACPTM | 94 | 0.5 |
| | ..S......R...... | 1 | |
| | ..............A. | 1 | |
| | .....E.......... | <1 | |
| | ..S..E...R...... | <1 | |
| | A.SATEI.SS...... | <1 | |
| | A............... | <1 | |
| | ..A............. | <1 | |
| | ..I............. | <1 | |
| | ..S............. | <1 | |
| | ......H......A.. | <1 | |
| | ......H...T..A.. | <1 | |
| | ........I....... | <1 | |
| | .............A.. | <1 | |
| | ...............V | <1 | |
| | .........N...... | <1 | |
| | S............... | <1 | |

^{a}Variant of the epitope sequence (in bold face) are shown with the variable amino acids.
^{b}Incidence of the epitope peptide sequence in all reported WNV sequence data. Those with <1% incidence do not include 0%.

### Example 5

### WNV-Specific T Cell Epitope Peptide Sequences

A notable finding was that, despite the high WNV protein conservation, only 51 of the 137 HLA-restricted WNV epitope peptides of this study were specific for WNV (Table 7), with the remaining 86 shared among a number of other flaviviruses. The concentration of WNV specific epitope sequences was greatest in the NS2a, NS2b, NS4a, and NS4b proteins, with 23 of the 30 total epitope peptides of these proteins were specific to WNV. In contrast, there were only 28 WNV specific peptides of the total of 107 epitope peptides present in the E, NS1 and NS3 proteins. The WNV specificity of the epitope peptides was not a function of the conservation, which ranged from 75 to 99% of the recorded sequences. Notably, none of the epitope peptides of NS5, which collectively were among the most highly conserved sequences, was WNV specific.

TABLE 7
WNV-specific epitope peptides.

| | | Inci- | |
|---|---|---|---|
| Protein & | | dence | En- |
| position | Peptide sequence | (%)^{a} | tropy |
| prM 136-153 | LVAAVIGWMLGSNTMQRV | 94 | 0.4 |
| E 62-77 | LATVSDLSTKAACPTM | 94 | 0.5 |
| E 119-136 | FACSTKAIGRTILKENIK | 81 | 0.9 |
| E 195-212 | GIDTNAYYVMTVGTKTFL | 86 | 0.9 |
| E 210-225 | TFLVHREWFMDLNLPW | 95 | 0.3 |
| E 356-373 | VTVNPFVSVATANAICVLI | 96 | 0.3 |
| E 384-399 | IVVGRGEQQINHHWHK | 99 | 0.1 |
| E 486-501 | LAVGGVLLFLSVNVHA | 98 | 0.2 |
| NS1 1-19 | DTGCAIDISRQELRCGSGV | 90 | 0.7 |
| NS1 161-178 | GLTSTRMFLKVRESNTTE | 87 | 0.9 |
| NS1 228-245 | THTLWGDGILESDLIIPV | 87 | 0.8 |
| NS2a 45-61 | VFGGITYTDVLRYVILV | 96 | 0.4 |
| NS2a 52-66 | TDVLRYVILVGAAFA | 97 | 0.3 |
| NS2a 80-97 | MATFKIQPVFMVASFLKA | 89 | 0.7 |
| NS2a 128-145 | EIPDVLNSLAVAWMILRA | 85 | 1.0 |
| NS2a 136-154 | LAVAWMILRAITFTTTSNV | 89 | 0.7 |
| NS2a 160-177 | ALLTPGLRCLNLDVYRIL | 87 | 0.8 |
| NS2a 168-183 | CLNLDVYRILLLMVGI | 78 | 1.0 |
| NS2a 211-229 | GLFNPMILAAGLIACDPNR | 75 | 1.3 |
| NS2b 59-75 | DISWESDAEITGSSERV | 85 | 0.9 |
| NS2b 84-101 | NFQLMNDPGAPWKIWMLR | 94 | 0.5 |
| NS2b 107-124 | ISAYTPWAILPSVVGFWI | 88 | 0.6 |
| NS2b 115-131 | ILPSVVGFWITLQYTKR | 89 | 0.6 |
| NS3 22-38 | VYRIMTRGLLGSYQAGA | 98 | 0.2 |
| NS3 50-65 | WHTTKGAALMSGEGRL | 99 | 0.1 |
| NS3 70-87 | GSVKEDRLCYGGPWKLQH | 97 | 0.3 |
| NS3 78-95 | CYGGPWKLQHKWNGQDEV | 87 | 0.8 |
| NS3 85-100 | LQHKWNGQDEVQMIVV | 88 | 0.7 |
| NS3 98-115 | IVVEPGICNVKNVQTKPGV | 97 | 0.3 |
| NS3 161-177 | YISAIVQGERMDEPIPA | 86 | 0.8 |
| NS3 176-192 | PAGFEPEMLRKKQITVL | 86 | 0.4 |
| NS3 241-258 | IRYQTSAVPREHNGNEIV | 81 | 1.1 |
| NS3 324-340 | DPFPESNSPISDLQTEI | 86 | 0.9 |
| NS3 474-489 | CYGGHTNEDDSNFAHW | 95 | 0.4 |
| NS3 480-496 | NEDDSNFAHWTEARIML | 94 | 0.4 |
| NS3 487-501 | AHWTEARIMLDNINM | 99 | 0.1 |
| NS3 492-509 | ARIMLDNINMPNGLIAQF | 89 | 0.6 |
| NS3 500-517 | NMPNGLIAQFYQPEREKV | 90 | 0.6 |
| NS3 581-597 | EVITKLGERKILRPRWI | 91 | 0.6 |
| NS3 588-605 | ERKILRPRWIDARVYSDH | 90 | 0.6 |
| NS4a 1-17 | SQIGLIEVLGKMPEHFM | 87 | 0.8 |
| NS4a 15-31 | HFMGKTWEALDTMYVVA | 97 | 0.2 |
| NS4a 54-71 | IALIALLSVMTMGVFFLL | 80 | 1.1 |
| NS4a 62-79 | VMTMGVFFLLMQRKGIGK | 92 | 0.5 |
| NS4a 86-103 | VLGVATFFCWMAEVPGTK | 84 | 1.0 |
| NS4b 38-55 | PATAWSLYAVTTAVLTPL | 97 | 0.2 |
| NS4b 63-80 | DYINTSLTSINVQASALF | 98 | 0.2 |
| NS4b 87-103 | PFVDVGVSALLLAAGCW | 96 | 0.3 |
| NS4b 201-218 | LITAAAVTLWENGASSVW | 91 | 0.5 |
| NS4b 233-250 | GWLSCLSITWTLIKNMEK | 76 | 1.5 |
| NS4b 241-255 | TWTLIKNMEKPGLKR | 79 | 1.3 |

^{a}Percentage incidence (round off to the nearest whole number) in all reported WNV sequence data. Those with >99% incidence do not include 100%. SEQ ID NOs for each peptide are identified in Table 2.

### Example 6

### Flavivirus-Shared WNV T-Cell Epitope Peptide Sequences

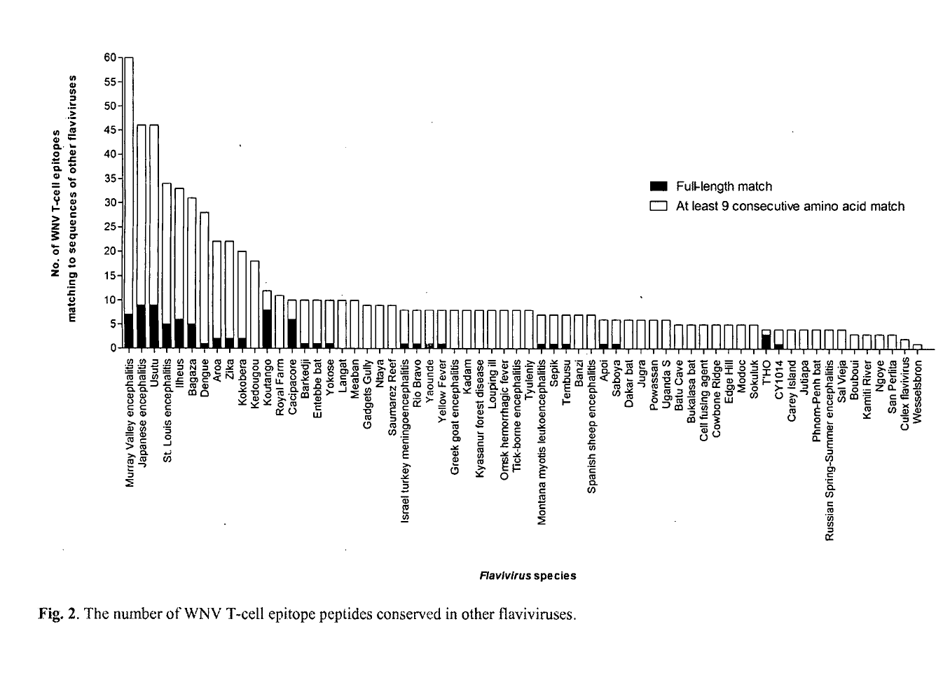

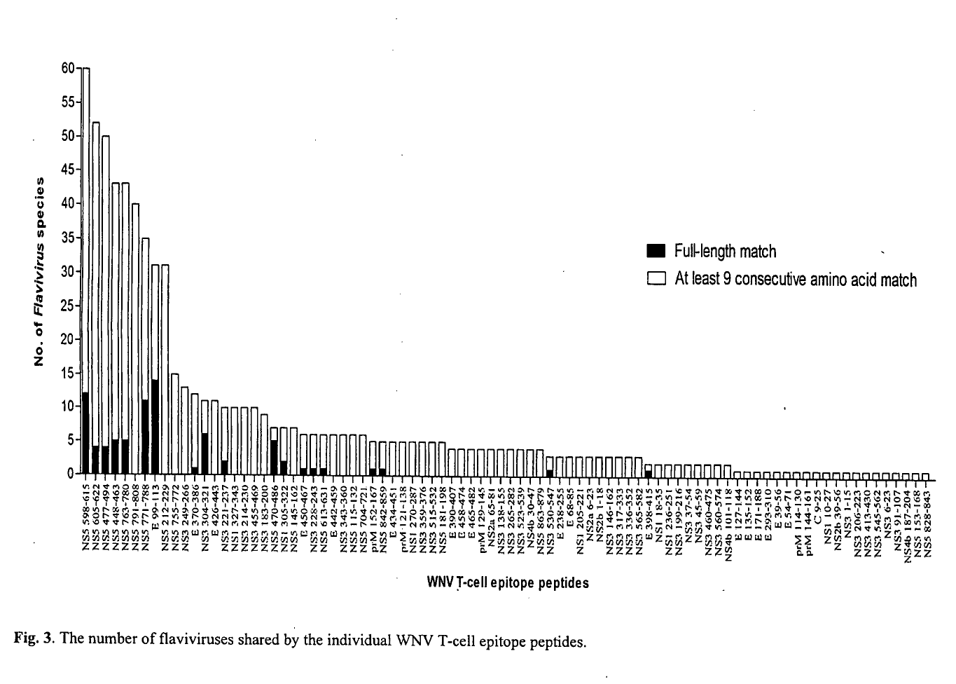

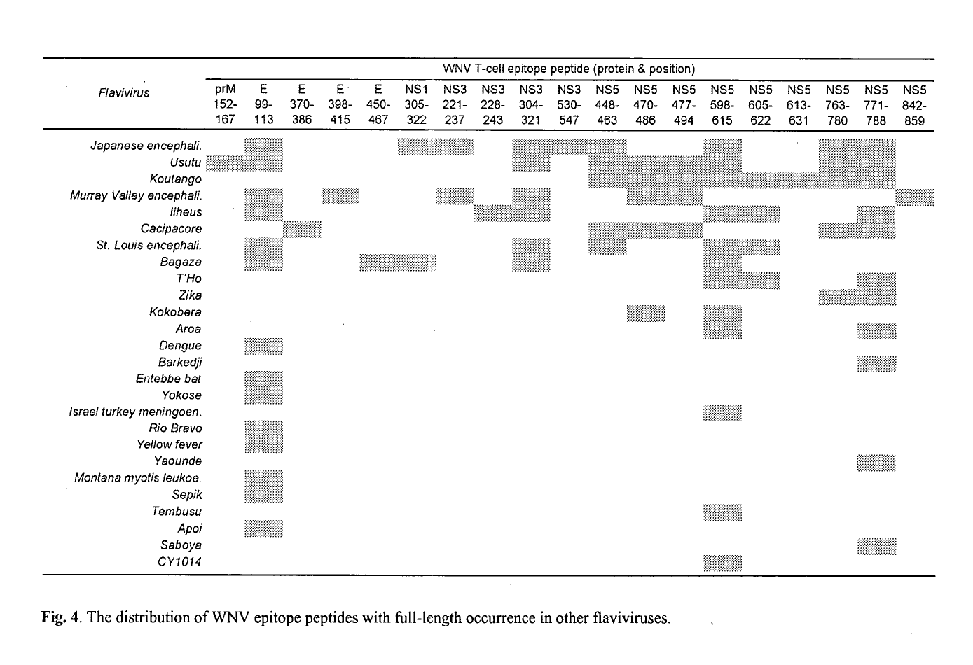

Representation of WNV epitope peptide sequences of 9 or more contiguous amino acids in other proteins was searched by BLAST analyses of all protein sequences deposited at NCBI (as of January 2009). Almost all flaviviruses, and few other viruses such as sindbis, Simian immunodeficiency and Trichoplusia ni SNPV, shared 1 or more such sequences of the 86 non-WNV specific epitope peptides, from a single WNV sequence in 1 Flavivirus to the presence of the NS5 598-615 sequence in 62 flaviviruses (FIGS. 2 and 3). Sequences of other NS5 epitope peptides and E 99-113 were each present in over 30 flaviviruses and particularly in the closely related Murray Valley encephalitis, Japanese encephalitis, and Usutu viruses. While most of the 86 non-WNV specific epitope peptides were only partially identical by sequences of 9 or more amino acids, 19 of the WNV epitope peptides were represented by complete sequence match in 28 other flaviviruses (Table 8). These complete epitope peptides consisted mainly of the E, NS3, and NS5 sequences, and included several of the most highly conserved sequences with entropy from 0.1 to 0.0. Three WNV epitope peptides (3) (E 99-113, NS5 598-615, and NS5 771-788) with entropies of 0.1 to 0.0 and conserved in >99% or all recorded WNV, were extensively represented in 12 to 14 other flaviviruses (FIG. 4). Japanese encephalitis and Usutu viruses each contained 9; Koutango, 8; Murray Valley, 7; Ilheus and Cacipacore, 6; St Louis and Bagaza, 5; and several others, 3 or fewer. There was no direct correlation between the diversity of the peptide and the number of flaviviruses that shared the sequence, and epitope peptides with greater entropies were also shared with multiple viruses. Because the 19 WNV epitope peptides were completely conserved in other flaviviruses, it is very likely that these shared sequences are corresponding HLA-restricted T cell epitopes of these viruses (FIG. 4).

TABLE 8
WNV T-cell epitope peptides with full-length
occurrence in other flaviviruses.

| | | Shared | | |
|---|---|---|---|---|
| Protein & | | flaviviruses | Incidence | |
| position | Peptide sequence | (#)^{a} | (%)^{b} | Entropy |
| prM 152-167 | RVVEVVLLLLVAPAYS | 1 | 95 | 0.4 |
| E 99-113 | RGWGNGCGLFGKGSI | 14 | >99 | 0.1 |
| E 370-386 | KVLIELEPPFGDSYIVV | 1 | >99 | 0.0 |
| E 398-415 | HKSGSSIGKAFTTTLKGA | 1 | 97 | 0.3 |
| E 450-467 | FRSLFGGMSWITQGLLGA | 1 | >99 | 0.1 |
| NS1 305-322 | GKLITDWCCRSCTLPPLR | 2 | 99 | 0.1 |
| NS3 221-237 | VLAPTRVVAAEMAEALR | 2 | 90 | 0.7 |
| NS3 228-243 | VAAEMAEALRGLPIRY | 1 | 90 | 0.6 |
| NS3 304-321 | KVELGEAAAIFMTATPPG | 6 | 95 | 0.3 |
| NS3 530-547 | ERKNFLELLRTADLPVWL | 1 | 92 | 0.4 |
| NS5 448-463 | ECHTCIYNMMGKREKK | 5 | 92 | 0.5 |
| NS5 470-486 | AKGSRAIWFMWLGARFL | 5 | >99 | 0.0 |
| NS5 477-494 | WFMWLGARFLEFEALGFL | 4 | 100 | 0.0 |
| NS5 598-615 | REDQRGSGQVVTYALNTF | 12 | 100 | 0.0 |
| NS5 605-622 | GQVVTYALNTFTNLAVQL | 4 | 99 | 0.1 |
| NS5 613-631 | NTFTNLAVQLVRMMEGEGV | 1 | 99 | 0.1 |
| NS5 763-780 | YAQMWLLLYFHRRDLRLM | 5 | >99 | 0.1 |
| NS5 771-788 | YFHRRDLRLMANAICSAV | 11 | 100 | 0.0 |
| NS5 842-859 | PYSGKREDIWCGSLIGTR | 1 | 98 | 0.1 |

^{a}Number of shared flaviviruses other than WNV.
^{b}Percentage incidence (round off to the nearest whole number) of the epitope peptide sequence in all reported WNV sequence data. Those with >99% incidence do not include 100%. SEQ ID NOs for each peptide are identified in Table 2.

### Example 7

### Representation of WNV T-Cell Epitope Peptides and their Variants in Major Flaviviruses

Further analysis of the sequence representation of the WNV epitope peptides was performed with other flaviviruses that had adequate database information. These included the Japanese encephalitis group [JEV, LEV (St. Louis EV)]; the tick-borne encephalitis virus group [TBEV, PV (powassan virus)]; yellow fever virus (YFV); and dengue virus (DV). The representation of many of the epitope peptides ranged from low (˜1%) to high (100%) among known sequences of the highly studied flaviviruses (Table 9). In particular, the epitopes from the NS5 protein were observed to be highly represented among many of the major flaviviruses, with identical or mutated sequences highly specific to the individual flaviviruses (Table 10). For example, the epitope peptide NS5₂₁₂₋₂₂₉ is present as the dominant sequence of the recorded WNV sequences (130 of 143) and not in any of the other selected viruses; however, specific mutant variants of this sequence were predominant peptides in LEV (26 or 29) and YFV (19 or 22); and several forms were present in dengue viruses with significant representation. The NS5 448-463 peptide and other WNV NS5 epitope peptides were either unique to WNV or shared with members of the closely related JEV group (LEV and/or JEV), and mutated forms were predominant peptide sequences in members of other less related flaviviruses of the tick-borne encephalitis virus group (TBEV and Powassan virus), yellow fever virus, and dengue. It thus is apparent that multiple forms of many WNV epitope peptide variants, that have only minor representation in the WNV database, are not specific for WNV and are widely present as predominant peptide sequences in other flaviviruses.

TABLE 9
The distribution of cross-reactive WNV T-cell epitope peptides in other major flaviviruses.

| | | Percentage incidence (%) ^{b} | | | | | |
|---|---|---|---|---|---|---|---|
| Protein & position | Peptide sequence ^{a} | LEV | JEV | TBEV | PV | YFV | DENV |
| prM 114-130 | STKATRYLVKTESWILR | | 0\|1 | | | | |
| prM 121-138 | LVKTESWILRNPGYALVA | 0\|100 | | | | | 0\|9 |
| prM 129-145 | LRNPGYALVAAVIGWML | 0\|100 | | | | | |
| prM 152-167 | RVVFVVLLLLVAPAYS | | 0\|97 | | | | |
| E 99-113 | RGWGNGCGLFGKGSI | 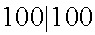 | 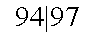 | 0\|>99 | | 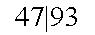 | 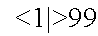 |
| E 127-144 | GRTILKENIKYEVAIFVH | 0\|98 | | | | | |
| E 135-152 | IKYEVAIFVHGPTTVESH | 0\|96 | | | | | |
| E 238-255 | TLMEFEEPHATKQSVIAL | 0\|98 | | | | | |
| E 293-310 | EKLQLKGTTYGVCSKAFK | | 0\|1 | | | | |
| E 370-386 | KVLIELEPPFGDSYIVV | 0\|93 | 0\|99 | | | | 0\|41 |
| E 426-443 | WDFGSVGGVFTSVGKAVH | | | | 0\|50 | | 0\|55 |
| E 434-451 | VFTSVGKAVHQVFGGAFR | 0\|99 | 0\|98 | | | | 0\|8 |
| E 442-459 | VHQVFGGAFRSLFGGMSW | 0\|98 | 0\|98 | | | | |
| E 450-467 | FRSLFGGMSWITQGLLGA | 0\|100 | 0\|99 | | | | |
| E 458-474 | SWITQGLLGALLLWMGI | 0\|100 | 0\|3 | | | | |
| E 465-482 | LGALLLWMGINARDRSIA | 0\|100 | 0\|4 | | | | |
| NS1 205-221 | RLNDTWKLERAVLGEVK | | 0\|94 | | | | |
| NS1 270-287 | EGRVEIDFDYCPGTTVTL | 0\|97 | 0\|2 | | | | |
| NS1 305-322 | GKLITDWCCRSCTLPPLR | | 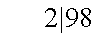 | | | | 0\|97 |
| NS1 327-343 | SGCWYGMEIRPQRHDEK | 0\|100 | 0\|98 | | | | 0\|100 |
| NS2a 6-23 | IDPFQLGLLVVFLATQEV | | 0\|98 | | | | |
| NS2b 1-18 | GWPATEVMTAVGLMFAIV | | 0\|94 | | | | |
| NS3 1-15 | GGVLWDTPSPKEYKK | | | | | | 0\|36 |
| NS3 6-23 | DTPSPKEYKKGDTTTGVY | | 0\|98 | | | | |
| NS3 37-54 | GAGVMVEGVFHTLWHTTK | | 0\|97 | | | | |
| NS3 45-59 | VFHTLWHTTKGAALM | | 0\|97 | | | | |
| NS3 138-155 | PIVDKNGDVIGLYGNGVI | | | | | | 0\|3 |
| NS3 146-162 | VIGLYGNGVIMPNGSYI | | | | | | 0\|3 |
| NS3 214-230 | NRRLRTAVLAPTRVVAA | 0\|100 | 0\|100 | | | | 0\|66 |
| NS3 221-237 | VLAPTRVVAAEMAEALR | 0\|100 | 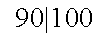 | | | | 0\|66 |
| NS3 228-243 | VAAEMAEALRGLPIRY | | 0\|95 | | | | 0\|41 |
| NS3 249-266 | PREHNGNEIVDVMCHATL | 0\|100 | 0\|100 | 0\|100 | | | |
| NS3 265-282 | TLTHRLMSPHRVPNYNLF | | 0\|100 | | | | |
| NS3 304-321 | KVELGEAAAIFMTATPPG | 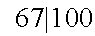 | 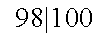 | | | | 0\|100 |
| NS3 343-360 | RAWNSGYEWITEYTGKTV | | 0\|75 | | | | 0\|24 |
| NS3 359-376 | TVWFVPSVKMGNEIALCL | 0\|100 | 0\|95 | | | | |
| NS3 455-469 | AAQRRGRIGRNPSQV | 0\|81 | | | | 0\|100 | 0\|73 |
| NS3 460-475 | GRIGRNPSQVGDEYCY | 0\|81 | 0\|2 | | | | |
| NS3 515-532 | EKVYTMDGEYRLRGEERK | | 0\|98 | | | | 0\|72 |
| NS3 523-539 | EYRLRGEERKNFLELLR | 0\|100 | 0\|2 | | | | |
| NS3 530-547 | ERKNFLELLRTADLPVWL | | 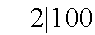 | | | | |
| NS3 545-562 | VWLAYKVAAAGVSYHDRR | | | | | | 0\|20 |
| NS3 560-574 | DRRWCFDGPRTNTIL | | 0\|100 | | | | |
| NS3 565-582 | FDGPRTNTILEDNNEVEV | 0\|100 | | | | | |
| NS4b 30-47 | GEFLLDLRPATAWSLYAV | | 0\|100 | | | | |
| NS4b 101-118 | GCWGQVTLTVTVTAATLL | | 0\|7 | | | | |
| NS5 145-162 | CDIGESSSSAEVEEHRTI | | | | | 0\|86 | |
| NS5 181-198 | VKVLCPYMPKVIEKMELL | 0\|64 | 0\|100 | | | | |
| NS5 212-229 | SRNSTHEMYWVSRASGNV | 0\|93 | | 0\|100 | 0\|100 | 0\|100 | 0\|99 |
| NS5 448-463 | ECHTCIYNMMGKREKK | 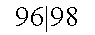 | 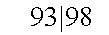 | 0\|100 | 0\|88 | 0\|100 | 0\|>99 |
| NS5 470-486 | AKGSRAIWFMWLGARFL | | 0\|100 | | | | |
| NS5 477-494 | WFMWLGARFLEFEALGFL | 0\|100 | 0\|100 | 0\|100 | | 0\|100 | 0\|>99 |
| NS5 598-615 | REDQRGSGQVVTYALNTF | 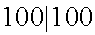 | 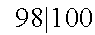 | 0\|100 | 0\|95 | 0\|100 | 0\|3 |
| NS5 605-622 | GQVVTYALNTFTNLAVQL | 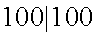 | 0\|100 | 0\|100 | 0\|90 | 0\|100 | |
| NS5 613-631 | NTFTNLAVQLVRMMEGEGV | 0\|100 | | | | | |
| NS5 704-721 | GWYDWQQVPFCSNHFTEL | | 0\|98 | | | | 0\|61 |
| NS5 755-772 | DTACLAKSYAQMWLLLYF | 0\|97 | 0\|95 | | | | 0\|<1 |
| NS5 763-780 | YAQMWLLLYFHRRDLRLM | | 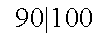 | | | | 0\|>99 |
| NS5 771-788 | YFHRRDLRLMANAICSAV | 0\|97 | 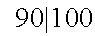 | | | | 0\|>99 |
| NS5 791-808 | NWVPTGRTTWSIHAGGEW | 0\|100 | | 0\|97 | 0\|100 | | |
| NS5 828-843 | WMEDKTPVEKWSDVPY | | | | | | 0\|34 |
| NS5 842-859 | PYSGKREDIWCGSLIGTR | 0\|100 | 0\|100 | | | | |
| NS5 863-879 | TWAENIQVAINQVRAII | | 0\|25 | | | | 0\|34 |

^{a} The WNV epitope peptide sequences that had 9 or more consecutive amino acids shared with any of the six other flaviviruses. WNV epitope peptide sequences that have a full-length match to the sequences of any one of the six other flaviviruses are shown in boldface and underlined.
^{b} Percentage incidence is depicted as “X|Y” where “X” refers to the percentage of the total virus sequences analyzed with full-length match to the WNV epitope peptide, and “Y” refers to the percentage of total virus sequences studied with ≧9 consecutive amino acids match to the WNV epitope. The “X” value is shaded 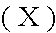 when there is a full-length match in the respective virus. The Flavivirus species abbreviations: LEV, St. Louis encephalitis virus; JEV, Japanese encephalitis virus; TBEV, Tick-borne encephalitis virus; PV, Powassan virus; YFV, Yellow fever virus and DENY, Dengue virus. SEQ ID NOs for each peptide are identified in Table 2.

TABLE 10
Variants of highly shared WNV epitope peptides and their
incidence in other selected flaviviruses. WNV variant
sequences representing less than about 10% of the
corresponding database sequences were omitted.

| | Representation ^{b} of the peptides in | | | | | | |
|---|---|---|---|---|---|---|---|
| WNV epitope peptide | the respective flaviviruses | | | | | | |
| and its variant ^{a} | (# of sequences analyzed) | | | | | | |
| | WNV | LEV | TBEV | PV | YFV | DENV | |
| NS5 212-229 | (143) | (29) | (29) | (21) | (22) | (1309) | Total |
| SRNSTHEMYWVSRASGNV | 130 | 0 | 0 | 0 | 0 | 0 | 130 |
| ............H....I | 11 | 0 | 0 | 0 | 0 | 0 | 11 |
| ............CGT..I | 0 | 0 | 0 | 0 | 0 | 471 | 471 |
| ..........I.NGT..I | 0 | 0 | 0 | 0 | 0 | 290 | 290 |
| ............N.T..I | 0 | 0 | 0 | 0 | 0 | 244 | 244 |
| ............N....I | 0 | 0 | 0 | 0 | 0 | 243 | 243 |
| ............G.A..I | 0 | 26 | 0 | 0 | 0 | 0 | 26 |
| .........Y..G.RS.. | 0 | 0 | 0 | 0 | 19 | 0 | 19 |
| | WNV | LEV | JEV | TBEV | PV | YFV | |
| NS5 448-463 | (182) | (45) | (60) | (33) | (24) | (22) | Total |
| ECHTCIYNMMGKREKK | 167 | 43 | 56 | 0 | 0 | 0 | 266 |
| Q.RH.V.......... | 0 | 0 | 0 | 0 | 21 | 0 | 21 |
| | WNV | LEV | JEV | TBEV | YFV | DENV | |
| NS5 477-494 | (181) | (48) | (60) | (33) | (23) | (1372) | Total |
| WFMWLGARFLEFRALGFL | 181 | 0 | 0 | 0 | 0 | 0 | 181 |
| .Y................ | 0 | 46 | 0 | 0 | 3 | 541 | 590 |
| .Y...............M | 0 | 0 | 0 | 0 | 0 | 474 | 474 |
| .Y......Y......... | 0 | 0 | 0 | 0 | 20 | 344 | 364 |
| ........Y......... | 0 | 0 | 60 | 0 | 0 | 0 | 60 |
| .Y....S........... | 0 | 0 | 0 | 33 | 0 | 0 | 33 |
| | WNV | LEV | JEV | TBEV | PV | DENV | |
| NS5 598-615 | (182) | (28) | (59) | (30) | (21) | (1379) | Total |
| REDQRGSGQVVTYALNTF | 182 | 28 | 58 | 0 | 0 | 0 | 268 |
| .K........G..G.... | 0 | 0 | 0 | 0 | 0 | 477 | 477 |
| | WNV | LEV | JEV | TBEV | PV | YFV | |
| NS5 605-622 | (182) | (28) | (59) | (30) | (21) | (22) | Total |
| GQVVTYALNTFTNLAVQL | 180 | 28 | 0 | 0 | 0 | 0 | 208 |
| ..............S... | 2 | 0 | 0 | 0 | 0 | 0 | 2 |
| ..........L..IK... | 0 | 0 | 0 | 30 | 0 | 0 | 30 |
| .............I.... | 0 | 0 | 58 | 0 | 0 | 0 | 58 |
| ..........I...K... | 0 | 0 | 0 | 0 | 0 | 21 | 21 |
| ..........I..MK... | 0 | 0 | 0 | 0 | 17 | 0 | 17 |

^{a} The epitope sequences are shown in bold face and the mutations in the variant peptides are shown by the respective variant amino acids.
^{b} Data was collected from the NCBI Entrez Protein Database (as of January 2009) SEQ ID NOs for each peptide are identified in Table 2.

TABLE 11
WNV HLA-restricted T-cell epitope peptides incidence and their variant
incidence distribution. Entropy describing the diversity at the epitope
peptide sites is also indicated.

| | | ELISpot positive peptides | | Variant peptides ^{c} | | |
|---|---|---|---|---|---|---|
| Protein & | | | Incidence | >10% | 1-10% | <1% |
| position | Entropy ^{a} | Peptide sequence | (%) ^{b} | (#) | (#) | (#) |
| C 9-25 | 0.7 | GKSRAVNMLKRGMPRVL | 88 | — | 1 | 5 |
| prM 114-130 | 0.1 | STKATRYLVKTESWILR | >99 | — | — | 3 |
| prM 121-138 | 0.1 | LVKTESWILRNPGYALVA | >99 | — | — | 2 |
| prM 129-145 | 0.3 | LRNPGYALVAAVIGWML | 95 | — | 1 | 2 |
| prM 136-153 | 0.4 | LVAAVIGWMLGSNTMQRV | 94 | — | 1 | 4 |
| prM 144-161 | 0.6 | MLGSNTMQRVVFVVLLLL | 92 | — | 3 | 4 |
| prM 152-167 | 0.4 | RVVFVVLLLLVAPAYS | 95 | — | 2 | 3 |
| E 39-56 | 0.3 | PTIDVKMMNMEAANLAEV | 96 | — | 1 | 9 |
| E 54-71 | 0.3 | AEVRSYCYLATVSDLSTK | 96 | — | 1 | 11 |
| E 62-77 | 0.5 | LATVSDLSTKAACPTM | 94 | — | 2 | 14 |
| E 68-85 | 0.4 | LSTKAACPTMGEAHNDKR | 95 | — | 2 | 9 |
| E 99-113 | 0.1 | RGWGNGCGLFGKGSI | >99 | — | — | 3 |
| E 119-136 | 0.9 | FACSTKAIGRTILKENIK | 81 | 1(16%) | 1 | 11 |
| E 127-144 | 0.3 | GRTILICENIKYEVAIFVH | 97 | — | 1 | 7 |
| E 135-152 | 0.1 | IKYEVAIFVHGPTTVESH | 99 | — | — | 8 |
| E 171-188 | 0.6 | PAAPSYTLKLGEYGEVTV | 91 | — | 2 | 12 |
| E 195-212 | 0.9 | GIDTNAYYVMTVGTKTFL | 86 | — | 2 | 11 |
| E 210-225 | 0.3 | TFLVHREWFMDLNLPW | 95 | — | 1 | 7 |
| E 238-255 | 0.3 | TLMEFEEPHATKQSVIAL | 97 | — | 1 | 8 |
| E 293-310 | 0.1 | EKLQLKGTTYGVCSKAFK | 99 | — | — | 6 |
| E 356-373 | 0.3 | VTVNPFVSVATANAKVLI | 96 | — | 1 | 8 |
| E 370-386 | 0 | KVLIELEPPFGDSYIVV | >99 | — | — | 2 |
| E 384-399 | 0.1 | IVVGRGEQQINHHWHK | >99 | — | — | 4 |
| E 390-407 | 0.1 | EQQINHHWHKSGSSIGKA | 99 | — | — | 6 |
| E 398-415 | 0.3 | HKSGSSIGKAFTTTLKGA | 97 | — | 1 | 5 |
| E 426-443 | 0.4 | WDFGSVGGVFTSVGKAVH | 95 | — | 2 | 5 |
| E 434-451 | 0.3 | VFTSVGKAVHQVFGGAFR | 96 | — | 1 | 5 |
| E 442-459 | 0.2 | VHQVFGGAFRSLFGGMSW | 97 | — | 1 | 2 |
| E 450-467 | 0.1 | FRSLFGGMSWITQGLLGA | >99.3 | — | — | 3 |
| E 458-474 | 0.1 | SWITQGLLGALLLWMGI | >99 | — | — | 4 |
| E 465-482 | 0.1 | LGALLLWMGINARDRSIA | 99 | — | — | 7 |
| E 486-501 | 0.2 | LAVGGVLLFLSVNVHA | 98 | — | — | 5 |
| NS1 1-19 | 0.7 | DTGCAIDISRQELRCGSGV | 90 | — | 1 | 5 |
| NS1 10-27 | 0.2 | RQELRCGSGVFIHNDVEA | 98 | — | — | 3 |
| NS1 18-35 | 0.7 | GVFIHNDVEAWMDRYKYY | 89 | — | 2 | 5 |
| NS1 161-178 | 0.9 | GLTSTRMFLKVRESNTTE | 87 | — | 3 | 6 |
| NS1 205-221 | 0.8 | RLNDTWKLERAVLGEVK | 87 | — | 3 | 2 |
| NS1 228-245 | 0.8 | THTLWGDGILESDLIIPV | 87 | — | 2 | 5 |
| NS1 236-251 | 0.7 | ILESDLIIPVTLAGPR | 89 | — | 2 | 5 |
| NS1 270-287 | 0.6 | EGRVEIDFDYCPGTTVTL | 91 | — | 2 | 2 |
| NS1 305-322 | 0.1 | GKLITDWCCRSCTLPPLR | 99 | — | — | 2 |
| NS1 327-343 | 0.8 | SGCWYGMEIRPQRHDEK | 87 | — | 3 | 4 |
| NS2a 6-23 | 0.4 | IDPFQLGLLVVFLATQEV | 94 | — | 1 | 3 |
| NS2a 45-61 | 0.4 | VFGGITYTDVLRYVILV | 96 | — | — | 6 |
| NS2a 52-66 | 0.3 | TDVLRYVILVGAAFA | 97 | — | — | 5 |
| NS2a 65-81 | 0.5 | FAESNSGGDVVHLALMA | 92 | — | 1 | 1 |
| NS2a 80-97 | 0.7 | MATFKIQPVFMVASFLKA | 89 | — | 1 | 5 |
| NS2a 128-145 | 1 | EIPDVLNSLAVAWMILRA | 85 | — | 4 | 5 |
| NS2a 136-154 | 0.7 | LAVAWMILRAITFTTTSNV | 88 | — | 1 | 5 |
| NS2a 160-177 | 0.8 | ALLTPGLRCLNLDVYRIL | 87 | — | 2 | 4 |
| NS2a 168-183 | 1 | CLNLDVYRILLLMVGI | 78 | 1(18%) | 1 | 4 |
| NS2a 211-229 | 1.3 | GLFNPMILAAGLIACDPNR | 75 | 1(12%) | 3 | 3 |
| NS2b 1-18 | 0.1 | GWPATEVMTAVGLMFAIV | >99 | — | — | 1 |
| NS2b 39-56 | 0.3 | MFAAFVISGKSTDMWIER | 94 | — | 1 | 1 |
| NS2b 59-75 | 0.9 | DISWESDAEITGSSERV | 85 | — | 3 | 3 |
| NS2b 84-101 | 0.5 | NFQLMNDPGAPWKIWMLR | 94 | — | 2 | 4 |
| NS2b 107-124 | 0.6 | ISAYTPWAILPSVVGFWI | 88 | — | 2 | 1 |
| NS2b 115-131 | 0.6 | ILPSVVGFWITLQYTKR | 89 | — | 1 | 2 |
| NS3 1-15 | 0.5 | GGVLWDTPSPKEYKK | 94 | — | 2 | 3 |
| NS3 6-23 | 0.5 | DTPSPKEYKKGDTTTGVY | 94 | — | 2 | 3 |
| NS3 22-38 | 0.2 | VYRIMTRGLLGSYQAGA | 98 | — | — | 3 |
| NS3 37-54 | 0.2 | GAGVMVEGVFHTLWHTTK | 97 | — | 1 | 2 |
| NS3 45-59 | 0.1 | VFHTLWHTTKGAALM | 99 | — | — | 2 |
| NS3 50-65 | 0.1 | WHTTKGAALMSGEGRL | 99 | — | — | 2 |
| NS3 70-87 | 0.3 | GSVKEDRLCYGGPWKLQH | 97 | — | 1 | 3 |
| NS3 78-95 | 0.8 | CYGGPWKLQHKWNGQDEV | 87 | — | 2 | 4 |
| NS3 85-100 | 0.7 | LQHKWNGQDEVQMIVV | 88 | — | 1 | 4 |
| NS3 91-107 | 0.7 | GQDEVQMIVVEPGKNVK | 88 | — | 1 | 4 |
| NS3 98-115 | 0.3 | IVVEPGKNVKNVQTKPGV | 97 | — | 1 | 3 |
| NS3 138-155 | 0.1 | PIVDKNGDVIGLYGNGVI | 99 | — | — | 2 |
| NS3 146-162 | 0.2 | VIGLYGNGVIMPNGSYI | 97 | — | — | 2 |
| NS3 161-177 | 0.8 | YISAIVQGERMDEPIPA | 86 | — | 2 | 3 |
| NS3 176-192 | 0.4 | PAGFEPEMLRKKQITVL | 95 | — | — | 7 |
| NS3 183-200 | 0.1 | MLRKKQITVLDLHPGAGK | 99 | — | — | 2 |
| NS3 199-216 | 0.6 | GKTRRILPQIIKEAINRR | 90 | — | 2 | 2 |
| NS3 206-223 | 0.6 | PQIIKEAINRRLRTAVLA | 91 | — | 1 | 4 |
| NS3 214-230 | 0.6 | NRRLRTAVLAPTRVVAA | 90 | — | 1 | 5 |
| NS3 221-237 | 0.7 | VLAPTRVVAAEMAEALR | 90 | — | 1 | 5 |
| NS3 228-243 | 0.6 | VAAEMAEALRGLPIRY | 90 | — | 2 | 2 |
| NS3 241-258 | 1.1 | IRYCITSAVPREHNGNEIV | 81 | — | 3 | 5 |
| NS3 249-266 | 1.1 | PREHNGNEIVDVMCHATL | 81 | — | 3 | 4 |
| NS3 265-282 | 0 | TLTHRLMSPHRVPNYNLF | 100 | — | — | — |
| NS3 304-321 | 0.3 | KVELGEAAAIFMTATPPG | 95 | — | 1 | 1 |
| NS3 317-333 | 0.7 | ATPPGTSDPFPESNSPI | 87 | 1(11%) | — | 4 |
| NS3 324-340 | 0.9 | DPFPESNSPISDLQTEI | 86 | — | 2 | 6 |
| NS3 336-352 | 0.6 | LQTEIPDRAWNSGYEWI | 90 | — | 2 | 3 |
| NS3 343-360 | 1.3 | RAWNSGYEWITEYTGKTV | 69 | 1(23%) | 2 | 1 |
| NS3 359-376 | 0.1 | TVWFVPSVKMGNEIALCL | 99 | — | — | 2 |
| NS3 413-430 | 0.2 | EMGANFKASRVIDSRKSV | 97 | — | — | 2 |
| NS3 455-469 | 0.4 | AAQRRGRIGRNPSQV | 95 | — | 2 | 2 |
| NS3 460-475 | 0.4 | GRIGRNPSQVGDEYCY | 95 | — | 2 | 2 |
| NS3 474-489 | 0.4 | CYGGHTNEDDSNFAHW | 95 | — | 2 | 1 |
| NS3 480-496 | 0.4 | NEDDSNFAHWTEARIML | 94 | — | 2 | 2 |
| NS3 487-501 | 0.1 | AHWTEARIMLDNINM | >99 | — | — | 1 |
| NS3 492-509 | 0.6 | ARIMLDNINMPNGLIAQF | 89 | — | 2 | 2 |
| NS3 500-517 | 0.6 | NMPNGLIAQFYQPEREKV | 90 | — | 2 | 1 |
| NS3 515-532 | 0.2 | EKVYTMDGEYRLRGEERK | 97 | — | — | 4 |
| NS3 523-539 | 0.5 | EYRLRGEERKNFLELLR | 92 | — | 1 | 2 |
| NS3 530-547 | 0.4 | ERKNFLELLRTADLPVWL | 92 | — | 1 | 1 |
| NS3 545-562 | 0.9 | VWLAYKVAAAGVSYHDRR | 87 | — | 2 | 5 |
| NS3 560-574 | 0.5 | DRRWCFDGPRTNTIL | 91 | — | 1 | 2 |
| NS3 565-582 | 0.1 | FDGPRTNTILEDNNEVEV | 99 | — | — | 2 |
| NS3 581-597 | 0.6 | EVITKLGERKILRPRWI | 91 | — | 1 | 4 |
| NS3 588-605 | 0.6 | ERKILRPRWIDARVYSDH | 90 | — | 1 | 3 |
| NS4a 1-17 | 0.8 | SQIGLIEVLGKMPEHFM | 87 | — | 2 | 3 |
| NS4a 15-31 | 0.2 | HFMGKTWEALDTMYVVA | 97 | — | 1 | 1 |
| NS4a 54-71 | 1.1 | IALIALLSVMTMGVFFLL | 80 | 1(11%) | 2 | 4 |
| NS4a 62-79 | 0.5 | VMTMGVFFLLMQRKGIGK | 92 | — | 1 | 2 |
| NS4a 86-103 | 1 | VLGVATFFCWMAEVPGTK | 84 | — | 4 | 4 |
| NS4b 30-47 | 0.5 | GEFLLDLRPATAWSLYAV | 92 | — | 1 | 3 |
| NS4b 38-55 | 0.2 | PATAWSLYAVTTAVLTPL | 97 | — | — | 4 |
| NS4b 63-80 | 0.2 | DYINTSLTSINVQASALF | 98 | — | 1 | 1 |
| NS4b 87-103 | 0.3 | PFVDVGVSALLLAAGCW | 96 | — | 1 | 2 |
| NS4b 101-118 | 0.8 | GCWGQVTLTVTVTAATLL | 87 | — | 2 | 4 |
| NS4b 187-204 | 0.6 | VVNPSVKTVREAGILITA | 90 | — | 1 | 5 |
| NS4b 201-218 | 0.5 | LITAAAVTLWENGASSVW | 91 | — | 1 | 3 |
| NS4b 233-250 | 1.5 | GWLSCLSITWTLIKNMEK | 76 | — | 5 | 5 |
| NS4b 241-255 | 1.3 | TWTLIKNMEKPGLKR | 79 | — | 4 | 5 |
| NS5 115-132 | 0.1 | LVQSYGWNIVTMKSGVDV | >99 | — | — | 1 |
| NS5 145-162 | 0.7 | CDIGESSSSAEVEEHRTI | 88 | — | 2 | 1 |
| NS5 153-168 | 0.7 | SAEVEEHRTIRVLEMV | 87 | — | 2 | 1 |
| NS5 181-198 | 0.6 | VKVLCPYMPKVIEKMELL | 91 | — | 1 | 4 |
| NS5 212-229 | 0.5 | SRNSTHEMYWVSRASGNV | 91 | — | 1 | 2 |
| NS5 448-463 | 0.5 | ECHTCIYNMMGKREKK | 92 | — | 2 | 1 |
| NS5 470-486 | 0.1 | AKGSRAIWFMWLGARFL | >99 | — | — | 1 |
| NS5 477-494 | 0 | WFMWLGARFLEFEALGFL | 100 | — | — | — |
| NS5 598-615 | 0 | REDQRGSGQVVTYALNTF | 100 | — | — | — |
| NS5 605-622 | 0.1 | GQVVTYALNTFTNLAVQL | 99 | — | 1 | — |
| NS5 613-631 | 0.1 | NTFTNLAVQLVRMMEGEGV | 99 | — | 1 | — |
| NS5 704-721 | 0 | GWYDWQQVPFCSNHFTEL | 100 | — | — | — |
| NS5 755-772 | 0.1 | DTACLAKSYAQMWLLLYF | >99 | — | — | 1 |
| NS5 763-780 | 0.1 | YAQMWLLLYFHRRDLRLM | >99 | — | — | 1 |
| NS5 771-788 | 0 | YFHRRDLRLMANAICSAV | 100 | — | — | — |
| NS5 791-808 | 0.7 | NWVPTGRTTWSIHAGGEW | 88 | — | 2 | 4 |
| NS5 828-843 | 1 | WMEDKTPVEKWSDVPY | 85 | — | 2 | 9 |
| NS5 842-859 | 0.1 | PYSGKREDIWCGSLIGTR | 98 | — | — | 3 |
| NS5 863-879 | 1.6 | TWAENIQVAINQVRAII | 71 | 1(12%) | 3 | 10 |

^{a} Entropy value indicating the diversity of the region in the protein alignment that contained the epitope peptide sequence.
^{b} The percentage of sequences (round off to the nearest whole number) analyzed that contained the exact sequence of the epitope peptide. Those with >99% do not include 100%
^{c} The fraction of the variants of the epitope peptide sequence, greater than 10%, 1-10%, and less than 1%. The actual percentage representation of the major variant sequence is shown for the 7 epitope peptides with a variant that represents greater than 10% of the WNV sequences analyzed (see also Table 4).

TABLE 12
List of highly conserved and specific sequences of Flavivirus species
West Nile virus (WNV), dengue virus (DENV), yellow fever virus (YFV) and
Japanese encephalitis virus (JEV). (SEQ ID NOs: 1-206, in the order as shown)

| Virus | Protein | Conserved Virus Specific Sequence |
|---|---|---|
| Dengue Virus (DENV) | E | VLGSQEGAMH |
| | NS1 | VHTWTEQYKFQ |
| | | AVHADMGYWIES |
| | | HTLWSNGVLES |
| | | GPWHLGKLE |
| | NS3 | GLYGNGVVT |
| | | LTIMDLHPG |
| | | LMRRGDLPVWL |
| | NS4a | QRTPQDNQL |
| | NS4b | PASAWTLYAVATT |
| | | HYAIIGPGLQAKATREAQKR |
| | | AAGIMKNPTVDGI |
| | | FWNTTIAVS |
| | NS5 | SGVEGEGLH |
| | | IFKLTYQNKVV |
| | | DQRGSGQVGTYGLNTFTNME |
| | | PTSRTTWSIHA |
| Japanese Encephalitis | C | SVAMKHLTSFK |
| Virus (JEV) | NS1 | GGITYTDLARYVVL |
| | NS2a | LDTYRIILL |
| | | MAKKKGAVL |
| | | GLALTSTGWFSPTTI |
| | NS2b | AAITGSSRRLDVKLD |
| | NS3 | GKLTPYWGSV |
| | | YGGPWRFDRKWNGT |
| | | DVQVIVVEPGK |
| | | SYVSAIVQG |
| | | LRGLPVRYQTSAVQREHQGN |
| | | ASAAQRRGRV |
| | | LDNIHMPNGLV |
| | | QLYGPEREKA |
| | NS4a | LATFFLWAAEV |
| | | GTKIAGTLL |
| | | ALLLMVVLIPEP |
| | | AANEYGMLE |
| | | SQAGSLFVLPRGVP |
| | | TDLDLTVGLV |
| | NS5 | ERENHLRGECHT |
| | | LARAIIELTY |
| | | EIVMKDGRS |
| | | RARISPGAG |
| Yellow Fever Virus (YFV) | C | MSGRKAQGKTLG |
| | prM | WCPDSMEYNCPNLS |
| | | GRMGERQLQ |
| | | ALLVLAVGPA |
| | E | KCVTVMAPDKPSLDISL |
| | | DLTLPWQSGS |
| | | HLVEFEPPH |
| | | VLIEVNPPFGDS |
| | | GDSRLTYQWHKEGSSI |
| | | RNMTMSMSMI |
| | NS1 | KRELKCGDG |
| | | KYSYYPEDPVKLASI |
| | | EEGKCGLNSVDSL |
| | | HEMWRSRADEINAI |
| | | YQRGTHPFSRIRDGLQYGWKTWGK |
| | | FSPGRKNGSFIIDGKSRKECPFSNRVWNS |
| | | ILGAAVNGKKSAHGSPTFWMGSHEVNGTWM |
| | | LDYKECEWP |
| | | THTIGTSVEE |
| | | MFMPRSIGGPVSSHN |
| | | IPGYKVQTNGPWMQVPLEV |
| | | CTMPPVSFHG |
| | | DGCWYPMEIRP |
| | NS2a | HAVPFGLVSMMIA |
| | | LLGAMLVGQVT |
| | | NNGGDAMYMALIA |
| | | GFGLRTLWSPRERLV |
| | | KDTSMQKTIP |
| | | GLTQPFLGLCA |
| | NS2b | SIPVNEALAA |
| | | GVLAGLAFQ |
| | | MENFLGPIAVGG |
| | | LMMLVSVAG |
| | | WEEEAEISGSS |
| | | EQGEFKLLSE |
| | | VMTSLALVGAA |
| | NS3 | SGDVLWDIPTPK |
| | | GIFQSTFLGASQ |
| | | TFLGASQRGVGVAQGGVFHTM |
| | | PSWASVKEDLVAYGGSWKL |
| | | WDGEEEVQLIAA |
| | | VVNVQTKPS |
| | | NGGEIGAVAL |
| | | YGNGILVGDNSFVSAISQTE |
| | | PGAGKTRRFLPQILAECARR |
| | | AECARRRLRTL |
| | | RRRLRTLVL |
| | | GLDVKFHTQAFSAHGSG |
| | | MCHATLTYRMLEPTR |
| | | AHFLDPASIAAR |
| | | ARGWAAHRARANESATILMTATPP |
| | | ATPPGTSDEFPHSNGEIEDVQTDIPSEPW |
| | | WILADKRPTAWFLP |
| | | AWFLPSIRA |
| | | LPSIRAANVMAASLRKAGK |
| | | IAEMGANLCV |
| | | KVAIKGPLRISA |
| | | GRIGRNPNRDGDSYYYSEPTSE |
| | | NAHHVCWLEASMLLDN |
| | | MLLDNMEVRGGMVAPLYG |
| | | KTPVSPGEMRLRDDQR |
| | | GLKTNDRKWCF |
| | | LRPRWCDERVSSDQSAL |
| | NS4a | RAYRNALSMMPEAMT |
| | NS4b | AAWTINVGI |
| | | LSPMLHHWIK |
| | | MLHHWIKVEYGNLSLSGI |
| | | QSASVLSFMDKG |
| | | PFMKMNISV |
| | | LILPGIKAQQSKL |
| | | DIEEAPEMP |
| | | LYEKKLALYL |
| | | KKLALYLLLALSL |
| | | SVAMCRTPFSL |
| | | PLIEGNTSLLWNGPMAVSMTGVMRGN |
| | NS5 | GKTLGEVWKRELNLLD |
| | | ARRHLAEGKV |
| | | HLAEGKVDT |
| | | AEGKVDTGVAVSRG |
| | | VAVSRGTAK |
| | | GTAKLRWFHERGYVKLEGRV |
| | | WCYYAAAQKE |
| | | YAAAQKEVSGVKG |
| | | EKWLACGVDNFC |
| | | EKLELLQRRFG |
| | | LLQRRFGGT |
| | | TFTVNQTSRLLMRRMRRPTGKVT |
| | | LPIGTRSVETDKGPL |
| | | DNDNPYRTWHYCGSY |
| | | IEEVTRMAMTD |
| | | RMAMTDTTP |
| | | KEKVDTRAKDPPAGTRKIM |
| | | VVNRWLFRHL |
| | | QWKTANEAVQDPKFWE |
| | | KLSEFGKAKG |
| | | EDHWASRENSGGG |
| | | GLQYLGYVI |
| | | EADLDDEQEI |
| | | MEMTYKNKVVK |
| | | YKNKVVKVLR |
| | | YALNTITNLKVQL |
| | | ALSHLNAMSK |
| | | MSKVRKDISEWQPSK |
| | | GRGRVSPGNGW |
| | | ACLSKAYANM |
| | | SKAYANMWSLMYFHKRDMRLLS |
| | | KRQDKLCGSL |
| West Nile Virus (WNV) | prM | LVAAVIGWMLGSNTMQRV |
| | E | LATVSDLSTKAACPTM |
| | | FACSTKAIGRTILKENIK |
| | | GIDTNAYYVMTVGTKTFL |
| | | TFLVHREWFMDLNLPW |
| | | VTVNPFVSVATANAKVLI |
| | | IVVGRGEQQINHHWHK |
| | | LAVGGVLLFLSVNVHA |
| | NS1 | DTGCAIDISRQELRCGSGV |
| | | RSVSRLEHQMW |
| | | GLTSTRMFLKVRESNTTE |
| | | THTLWGDGILESDLIIPV |
| | NS2a | VFGGITYTDVLRYVILV |
| | | TDVLRYVILVGAAFA |
| | | MATFKIQPVFMVASFLKA |
| | | EIPDVLNSLAVAWMILRA |
| | | LAVAWMILRAITFTTTSNV |
| | | ALLTPGLRCLNLDVYRIL |
| | | CLNLDVYRILLLMVGI |
| | | GLFNPMILAAGLIACDPNR |
| | NS2b | GWPATEVMTA |
| | | PMTIAGLMF |
| | | DISWESDAEITGSSERV |
| | | NFQLMNDPGAPWKIWMLR |
| | | ISAYTPWAILPSVVGFWI |
| | | ILPSVVGFWITLQYTKR |
| | NS3 | VYRIMTRGLLGSYQAGA |
| | | WHTTKGAALMSGEGRLDPYWGSV |
| | | GSVKEDRLCYGGPWKLQH |
| | | CYGGPWKLQHKWNGQDEV |
| | | LQHKWNGQDEVQMIVV |
| | | IVVEPGKNVKNVQTKPGV |
| | | YISAIVQGERMDEPIPA |
| | | PAGFEPEMLRKKQITVL |
| | | IRYQTSAVPREHNGNEIV |
| | | DPFPESNSPISDLQTEIPDRAWN |
| | | RVIDSRKSVKP |
| | | CYGGHTNEDDSNFAHW |
| | | NEDDSNFAHWTEARIML |
| | | AHWTEARIMLDNINM |
| | | ARIMLDNINMPNGLIAQF |
| | | NMPNGLIAQFYQPEREKV |
| | | EVITKLGERKILRPRWI |
| | | ERKILRPRWIDARVYSDH |
| | NS4a | SQIGLIEVLGKMPEHFM |
| | | HFMGKTWEALDTMYVVA |
| | | IALIALLSVMTMGVFFLL |
| | | VMTMGVFFLLMQRKGIGK |
| | | VLGVATFFCWMAEVPGTK |
| | NS4b | PATAWSLYAVTTAVLTPL |
| | | DYINTSLTSINVQASALF |
| | | PFVDVGVSALLLAAGCW |
| | | LITAAAVTLWENGASSVW |
| | | GWLSCLSITWTLIKNMEK |
| | | TWTLIKNMEKPGLKR |
| | NS5 | GGAKGRTLGE |
| | | VEDWLHRGP |
| | | EREAHLRGEC |

### Example 8

Large-scale analysis of the T-cell epitopes of WNV by immunization of HLA transgenic mice with 452 overlapping peptides spanning the entire WNV proteome has resulted in the identification of 137 peptides that elicited 200 HLA-restricted IFN-γ T-cell responses in 6 HLA transgenic mice strains: 74 for class I A2, A24, and B7, and 126 for class II DR2, DR3, and DR4. The multiple HLA responses to some of the peptides can be attributed to peptide promiscuity in T-cell activation, and to multiple T-cell epitope sequences in the same peptide. Many of these T-cell epitope peptides are likely dominant immunogens in nature, whether conveyed by natural pathogens or vaccines. Several mechanism(s) by which exogenous peptide immunogens are presented by antigen presenting cells in both HLA class I and II pathways have been described (Ackerman and Cresswell, 2004; Giodini and Cresswell, 2008; Lindner and Unanue, 1996; Nygard et al., 1994; Pathak and Blum, 2000; Pinet et al., 1995). Mechanistic studies have also shown that exogenous peptides compete for the presentation of endogenous antigens to MHC II-restricted T cells (Adorini et al., 1991) and that both endogenously processed peptide and the corresponding exogenous peptide act as ligands for a T-cell receptor (Gyotoku et al., 1998). There is also an abundance of evidence that support the HLA-transgenic mouse model for the efficient identification of peptides that contain sequences recognized both by the HLA molecules of the transgenic mice and by antigen receptors of the mouse T cells (Sonderstrup et al., 1999; Taneja and David, 1999). However, a limitation of this and most studies of T-cell epitopes is that because of the complexity of peptide HLA-processing and T-cell receptor recognition, the specific minimal epitope sequences are not known. As pointed out by Niels Jerne in 1960 (Jerne, 1960) processed peptides that would be recognized by T cells in association with MHC molecules are what he termed “cryptotopes,” hidden epitopes which become immunologically available only after cellular processing. “T cell epitopes” as is now commonly used, describes the peptide sequence of the original protein, not the form that it is recognized by the T cell. For this reason, we herein use the terms “T cell epitope peptide” or “T cell epitope determinant” to describe the 15-18 amino acid peptides that contain T-cell epitopes of unknown specific sequence. Moreover, this mouse data is not used to elucidate the functional properties of human T-cells because the mouse T cells are educated to the HLA transgene, and little is known of the nature of this response as compared to the response of naive human T cells. Thus, our basic interpretation of these studies is that they reveal WNV protein sequences that contain T-cell epitopes specific for the selected HLA molecules and T cell class I or class II activation, but not a more detailed understanding of the functional role of these sequences in pathogen infection of humans.

A remarkable finding was the extensive identity of WNV epitope peptide sequences with other flaviviruses. WNV are among the more highly conserved RNA viruses with an average peptide sequence conservance of about 92% in all WNV in the public databases. In this study, there were only 7 epitope peptides that differed in more than 10% of the corresponding database sequences. However, and importantly, only 51 of the 137 epitope peptide sequences were specific for WNV and the remaining 86 contained sequences of 9 or more amino acids that collectively were identical to at least 67 other flaviviruses. Moreover, the entire sequences of 19 WNV epitope peptides, chiefly of the E, NS3 and NS5 proteins, were present in 26 viruses. Additionally, immune relevant homologous sequences of 9 or more amino acids of the 86 shared WNV epitope peptides were commonly found in other flaviviruses, with as many as 45 to 62 WNV sequences in Murray Valley encephalitis, Japanese encephalitis, and Usutu viruses. These mainly included E 99-113 and/or representatives of 8 NS5 sequences that each was found in 32 to 62 other flaviviruses. While the data presented are specific for WNV and other similar flaviviruses, we expect that the same would be true to some extent among other groups of phylogenetically related flaviviruses, certainly the 4 serotypes of dengue which contain many sequences with inter-DENV identity (Khan et al., 2008). There is high probability that many of the homologous sequences would act as epitopes or as altered peptide ligands in the event of multiple Flavivirus infections or immunization followed by infection with similar flaviviruses.

These findings have strong bearing on the possible pathological consequences of exposure to altered peptide ligands. Many studies have shown that peptide analogs recognized by T-cell receptors are participants in many T cell biological phenomena with possible pathogenic consequences (reviewed in Sloan-Lancaster and Allen, 1996; Mongkolsapaya, J. 2003). The selection of evolutionarily conserved protein sequences has widely been considered important to vaccine design in order to limit the selective loss of immunity resulting from mutation and protein modification. However, as shown herein, in the evolution of viruses, conserved sequences can be present in many different forms in viruses of related species and our conclusion is that the selection of virus specific sequences should have precedance to conserved, non-virus specific sequences. These observations also question the use of “ChimeriVax” vaccines such as the use of a yellow fever virus vaccine platform to deliver the premembrane and envelope genes of WNV (Monath et al., 2006), which clearly would have the potential of exposing the vaccine recipient to a large number of altered peptide ligands in the event of infection by WNV or any other Flavivirus.

The methodology applied to this study of WNV sequences provides an experimental basis for identification of HLA-restricted T cell epitope peptides of any pathogen. The analysis of pathogen antigens may conveniently use the same overlapping peptides required for ELISpot analysis of peptide specific T cell activation, but experiments comparing peptide and DNA-encoded antigen shown in this study and other unpublished experiments uniformly suggest the preferred use of genetic immunogens as vaccines. Selection of T-cell epitope peptides for vaccine design would omit sequences that are highly conserved in other related viruses, and focus on pathogen-specific sequences present in 80% or more of all recorded evolutionary variants of the pathogen and have clustered or closely contiguous localization. Clustered epitopes has distinct advantages in the design of an epitope-based vaccine, including the retention of native sequences for the function of transporters associated with antigen processing (TAPs) (Niedermann, 2002) and for the flanking sequences that are reported to modulate epitope processing and function in the selection of immunodominant epitopes (Le Gall et al., 2007). We elsewhere describe the use of sequences of the lysososome-associated membrane protein (LAMP) in the vaccine construct to elicit enhanced antigen delivery to the MHC II compartment of antigen presenting cells (de Arruda et al., 2004; Marques et al., 2003; Ruff et al., 1997).

### REFERENCES

The disclosure of each reference cited is expressly incorporated herein.
Ackerman, A. L. and Cresswell, P. (2004). Cellular mechanisms governing cross-presentation of exogenous antigens, Nat Immunol, 5, 678-84.
Adorini, L., Moreno, J., Momburg, F., Hammerling, G. J., Guery, J. C., Valli, A. and Fuchs, S. (1991). Exogenous peptides compete for the presentation of endogenous antigens to major histocompatibility complex class II-restricted T cells, J Exp Med, 174, 945-8.
Altschul, S. F., Gish, W., Miller, W., Myers, E. W. and Lipman, D. J. (1990). Basic local alignment search tool, J Mol Biol, 215, 403-10.
BenMohamed, L., Krishnan, R., Longmate, J., Auge, C., Low, L., Primus, J. and Diamond, D. J. (2000). Induction of CTL response by a minimal epitope vaccine in HLA A*0201/DR1 transgenic mice: dependence on HLA class II restricted T(H) response, Hum Immunol, 61, 764-79.
Berman, H. M., Westbrook, J., Feng, Z., Gilliland, G., Bhat, T. N., Weissig, H., Shindyalov, I. N. and Bourne, P. E. (2000). The Protein Data Bank, Nucleic Acids Res, 28, 235-42.
Blaney, J. E., Jr., Nobusawa, E., Brehm, M. A., Bonneau, R. H., Mylin, L. M., Fu, T. M., Kawaoka, Y. and Tevethia, S. S. (1998). Immunization with a single major histocompatibility complex class I-restricted cytotoxic T-lymphocyte recognition epitope of herpes simplex virus type 2 confers protective immunity, J Virol, 72, 9567-74.
Brien, J. D., Uhrlaub, J. L. and Nikolich-Zugich, J. (2007). Protective capacity and epitope specificity of CD8(+) T cells responding to lethal West Nile virus infection, Eur J Immunol, 37, 1855-63.
Castrucci, M. R., Hou, S., Doherty, P. C. and Kawaoka, Y. (1994). Protection against lethal lymphocytic choriomeningitis virus (LCMV) infection by immunization of mice with an influenza virus containing an LCMV epitope recognized by cytotoxic T lymphocytes, J Virol, 68, 3486-90.
Cheuk, E., D'Souza, C., Hu, N., Liu, Y., Lang, H. and Chamberlain, J. W. (2002). Human MHC class I transgenic mice deficient for H2 class I expression facilitate identification and characterization of new HLA class I-restricted viral T cell epitopes, J Immunol, 169, 5571-80.

Cope, A. P., Patel, S. D., Hall, F., Congia, M., Hubers, H. A., Verheijden, G. F., Boots, A. M., Menon, R., Trucco M., Rijnders, A. W. and Sonderstrup. G. (1999). T cell responses to a human cartilage autoantigen in the context of rheumatoid arthritis-associated and nonassociated HLA-DR4 alleles, Arthritis Rheum, 42, 1497-507.
Davis, C. T., Galbraith, S. E., Zhang, S., Whiteman, M. C., Li, L., Kinney, R. M. and Barrett, A. D. (2007). A combination of naturally occurring mutations in North American West Nile virus nonstructural protein genes and in the 3′ untranslated region alters virus phenotype, J Virol, 81, 6111-6.
de Arruda, L. B., Chikhlikar, P. R., August, J. T. and Marques, E. T. (2004). DNA vaccine encoding human immunodeficiency virus-1 Gag, targeted to the major histocompatibility complex II compartment by lysosomal-associated membrane protein, elicits enhanced long-term memory response, immunology, 112, 126-33.
de la Rosa, G., Yanez-Mo, M., Samaneigo, R., Serrano-Gomez, D., Martinez-Munoz, L., Fernandez-Ruiz, E., Longo, N., Sanchez-Madrid, F., Corbi, A. L. and Sanchez-Mateos, P. (2005). Regulated recruitment of DC-SIGN to cell-cell contact regions during zymosan-induced human dendritic cell aggregation, J Leukoc Biol, 77, 699-709.
Del Val, M., Schlicht, H. J., Volkmer, H., Messerle, M., Reddehase, M. J. and Koszinowski, U. H. (1991). Protection against lethal cytomegalovirus infection by a recombinant vaccine containing a single nonameric T-cell epitope, J Virol, 65, 3641-6.
Edgar, R. C. (2004). MUSCLE: multiple sequence alignment with high accuracy and high throughput, Nucleic Acids Res, 32, 1792-7.
Ferrante, A. and Gorski, J. (2007). Cooperativity of hydrophobic anchor interactions: evidence for epitope selection by MHC class II as a folding process, J Immunol, 178, 7181-9.
Fugger, L., Michie, S. A., Rulifson, I., Lock, C. B. and McDevitt, G. S. (1994). Expression of HLA-DR4 and human CD4 transgenes in mice determines the variable region beta-chain T-cell repertoire and mediates an ULA-DR-restricted immune response, Proc Natl Acad Sci S A, 9/, 6151-5.
Giodini, A. and Cresswell, P. (2008). Hsp90-mediated cytosolic refolding of exogenous proteins internalized by dendritic cells, EMBO J, 27, 201-11.
Goodridge, H. S., Simmons, R. M. and Underhill, D. M. (2007). Dectin-1 stimulation by Candida albicans yeast or zymosan triggers NEAT activation in macrophages and dendritic cells, J Immunol, 178, 3107-15.
Gyotoku, T., Fukui, Y. and Sasazuki, T. (1998). An endogenously processed self peptide and the corresponding exogenous peptide bound to the same MHC class II molecule could be distinct ligands for TCR with different kinetic stability, Eur J Immunol, 28, 4050-61.
Hu, N., D'Souza, C., Cheung, H., Lang, H., Cheuk, E. and Chamberlain, J. W. (2005). Highly conserved pattern of recognition of influenza A wild-type and variant CD8+ CTL epitopes in HLA-A2+ humans and transgenic HLA-A2+/H2 class I-deficient mice. Vaccine, 23, 5231-44.
Jerre, N. K. (1960). Immunological speculations, Annu Rev Microbiol, 14, 341-58.
Kessler, P. D., Podsakoff, G. M., Chen, X., McQuiston, S. A., Colosi, P. C., Matelis, L. A., Kurtzman, G. J. and Byrne, B. J. (1996). Gene delivery to skeletal muscle results in sustained expression and systemic delivery of a therapeutic protein., Proc Natl Acad Sci USA, 93, 14082-7.
Khan, A. M., Miotto, O., Nascimento, E. J., Srinivasan, K. N., Heiny, A. T., Zhang, G. L., Marques, E. T., Tan, T. W., Brusic, V., Salmon, J. and August, J. T. (2008). Conservation and variability of dengue virus proteins: implications for vaccine design, PLoS Negl Trop Dis, 2, e272.
Kuno, G., Chang, G. J., Tsuchiya, K. R., Karabatsos, N. and Cropp, C. B. (1998). Phylogeny of the genus Flavivirus, J Virol, 72, 73-83.
La. Posta, V. J., Auperin, D. D., Kamin-Lewis, R. and Cole, G. A. (1993). Cross-protection against lymphocytic choriomeningitis virus mediated by a CD4+ T-cell clone specific for an envelope glycoprotein epitope of Lassa virus, J Virol, 67, 3497-506.
Le Gall, S., Stamegna, P. and Walker, B. D. (2007). Portable flanking sequences modulate CTL epitope processing, J Clin invest, 117, 3563-75.
Lieberman, M. M., Clements, D. E., Ogata, S., Wang, G., Corpuz, G., Wong, T., Martyak, T., Gilson, L., Coller, B. A., Leung, J., Watts, D. M., Tesh, R. B., Siirin, M., Travassos da Rosa, A., Humphreys, T. and Weeks-Levy, C. (2007). Preparation and immunogenic properties of a recombinant West Nile subunit vaccine, Vaccine, 25, 414-23.
Lindner, R. and Unanue, E. R. (1996). Distinct antigen MHC class II-complexes generated by separate processing pathways, EMBO J, 15, 6910-20.
Loirat, D., Lemonnier, F. A. and Michel, M. L. (2000). Multiepitopic HLA-A*0201-restricted immune response against hepatitis B surface antigen after DNA-based immunization, J Immunol, 165, 4748-55.
Madsen, L., Labrecque, N., Engberg, J., Dicrich, A., Svejgaard, A., Benoist, C., Mathis, D. and Fugger, L. (1999). Mice lacking all conventional MHC class II genes, Proc Natl Acad Sci USA, 96, 10338-43.
Marques, E. T., Jr., Chikhlikar, P., de Arruda, L. B., Leao, I. C., Lu, Y., Wong, J., Chen, J. S., Byrne, B. and August, J. T. (2003). HIV-1 p55Gag encoded in the lysosome-associated membrane protein-1 as a DNA plasmid vaccine chimera is highly expressed, traffics to the major histocompatibility class II compartment, and elicits enhanced immune responses, J Biol Chem, 278, 37926-36.
McMurtrey, C. P., Lelic, A., Piazza, P., Chakrabarti, A. K., Yablonsky, E. J., Wahl, A., Bardet, W., Eckerd, A., Cook, R. L., Hess, R., Buchli, R., Loeb, M., Rinaldo, C. R., Bramson, J. and Hildebrand, W. H. (2008). Epitope discovery in West Nile virus infection: Identification and immune recognition of viral epitopes, Proc Natl Acad Sci USA, 105, 2981-6.
Miotto, O., Tan, T. W. and Brusic, V. (2008). Rule-based knowledge aggregation for large-scale protein sequence analysis of influenza A viruses, BMC Bioinformatics, 9 Suppl 1, S7.
Monath, T. P., Liu, J., Kanesa-Thasan, N., Myers, G. A., Nichols, R., Deary, A., McCarthy, K., Johnson, C., Ermak, T., Shin, S., Arroyo, J., Guirakhoo, F., Kennedy, J. S., Ennis, F. A., Green, S. and Bedford, P. (2006). A live, attenuated recombinant West Nile virus vaccine, Proc Natl Acad Sci USA, 103, 6694-9.
Mongkolsapaya, J., Dejnirattisai, W., Xu, X. N., Vasanawathana, S., Tangthawornchaikui, N., Chairunsri, A., Sawasdivorn, S., Duangchinda, T., Dong, T., Rowland-Jones, S., Yenchitsomanus, P. T., McMichael, A., Malasit, P. and Screaton, G. (2003). Original antigenic sin and apoptosis in the pathogenesis of dengue hemorrhagic fever, Nat Med, 9, 921-7.
Mongkolsapaya, J., Duangchinda, T., Dejnirattisai, W., Vasanawathana, S., Avirutnan., P., Jairungsri, A., Khemnu, N., Tangthawornchaikul, N., Chotiyarnwong, P., Sae-Jang, K., Koch, M., Jones, Y., McMichael, A., Xu, X., Malasit, P. and Screaton, G. (2006). T cell responses in dengue hemorrhagic fever: are cross-reactive T cells suboptimal?, J Immunol, 176, 3821-9.
Moudy, R. M., Meola, M. A., Morin, L. L., Ebel, G. D. and Kramer, L. D. (2007). A newly emergent genotype of West Nile virus is transmitted earlier and more efficiently by Culex mosquitoes, Am J Trop Med Hyg, 77, 365-70.
Niedermann, C. (2002). Immunological functions of the proteasome, Curr Top Microbiol Immunol, 268, 91-136.
Nygard, N. R., Giacoletto, K. S., Bono, C., Gorka, J., Kompelli, S. and Schwartz, B. D. (1994). Peptide binding to surface class II molecules is the major pathway of formation of immunogenic class II-peptide complexes for viable antigen presenting cells, J Immunol, 152, 1082-93.
Oukka, M., Manuguerra, J. C., Livaditis, N., Tourdot, S., Riche, N., Vergnon, I., Cordopatis, P. and Kosmatopoulos, K. (1996). Protection against lethal viral infection by vaccination with nonimmunodominant peptides, J Immunol, 157, 3039-45.
Pajot, A., Michel, M. L., Fazillcau, N., Pancre, V., Auriault, C., Ojcius, D. M., Lemonnier, F. A. and Lone, Y. C. (2004). A mouse model of human adaptive immune functions: HLA-A2.1-/HLA-DR1-transgenic H-2 class I-/class II-knockout mice, Eur J Immunol, 34, 3060-9.
Pajot, A., Michel, M. L., Mancini-Bourgine, M., Ungeheuer, M. N., Ojcius, D. M., Deng, Q., Lemonnier, F. A. and Lone, Y. C. (2006). Identification of novel HLA-DR1-restricted epitopes from the hepatitis B virus envelope protein in mice expressing HLA-DR1 and vaccinated human subjects, Microbes infect, 8, 2783-90.
Parsons, R., Lelic, A., Hayes, L., Carter, A., Marshall, L., Evelegh, C., Drebot, M., Andonova, M., McMurtrey, C., Hildebrand, W., Loeb, M. B. and Bramson, J. L. (2008). The memory T cell response to West Nile virus in symptomatic humans following natural infection is not influenced by age and is dominated by a restricted set of CD8+ T cell epitopes, J Immunol, 181, 1563-72.
Pascolo, S. (2005). HLA class I transgenic mice: development, utilisation and improvement, Expert Opin Biol Ther, 5, 919-38.
Pascolo, S., Bervas, N., tire, J. M., Smith, A. G., Lemonnier, F. A. and Perarnau, B. (1997). HLA-A2.1-restricted education and cytolytic activity of CD8(+) T lymphocytes from betα2 microglobulin (beta2m) HLA-A2.1 monochain transgenic H-2 Db beta2m double knockout mice, J Exp Med, 185, 2043-51.
Pathak, S. S. and Blum, J. S. (2000). Endocytic recycling is required for the presentation of an exogenous peptide via MHC class II molecules, Traffic, 1, 561-9.
Pinet, V., Vergelli, M., Martin, R., Bakke, O. and Long, E. O. (1995). Antigen presentation mediated by recycling of surface HLA-DR molecules, Nature, 375, 603-6.
Purtha, W. E., Myers, N., Mitaksov, V., Sitati, E., Connolly, J., Fremont, D. H., Hansen, T. H. and Diamond, M. S. (2007). Antigen-specific cytotoxic T lymphocytes protect against lethal West Nile virus encephalitis, Eur J Immunol, 37, 1845-54.
Richards, K. A., Chaves, F. A., Krafcik, F. R., Topham, D. J., Lazarski, C. A. and Sant, A. J. (2007). Direct ex vivo analyses of HLA-DR1 transgenic mice reveal an exceptionally broad pattern of immunodominance in the primary HLA-DR1-restricted CD4 T-cell response to influenza virus hemagglutinin, J Virol, 81, 7608-19.
Robinson, J., Waller, M. J., Fail, S. C. and Marsh, S. G. (2006). The IMGT/HLA and IPD databases, Hum Mutat, 27, 1192-9.
Roederer, M. and Koup, R. A. (2003). Optimized determination of T cell epitope responses, J Immunol Methods, 274, 221-8.
Rohrlich, P. S., Cardinaud, S., Firat, Lamari, M., Briand, P., Escriou, N. and Lemonnier, F. A. (2003). HLA-B*0702 transgenic, H-2 KbDb double-knockout mice: phenotypical and functional characterization in response to influenza virus, Int Immunol, 15, 765-72.
Rothman, A. L. (2004). Dengue: defining protective versus pathologic immunity, J Clin Invest, 113, 946-51.
Ruff, A. L., Guarnieri, F. G., Staveley-O'Carroll, K., Siliciano, R. F. and August, J. T. (1997). The enhanced immune response to the HIV gp160/LAMP chimeric gene product targeted to the lysosome membrane protein trafficking pathway, J Biol Chem, 272, 8671-8.
Screaton, G. and Mongkolsapaya, J. (2006). T cell responses and dengue haemorrhagic fever, Novartis Found Symp, 277, 164-71; discussion 171-6, 251-3.
Shannon, C. E. (1948). A mathematical theory of communication, Bell System Technical Journal, 27, 379-423 and 623-656.
Sloan-Lancaster, J. and Allen, P. M. (1996). Altered peptide ligand-induced partial T cell activation: molecular mechanisms and role in T cell biology, Annu Rev Immunol, 14, 1-27.
Sonderstrup, G., Cope, A. P., Patel, S., Congia, M., Hain, N., Hall, F. C., Parry, S. L., Fugger, L. H., Michie, S. and McDevitt, H. O. (1999). HLA class II transgenic mice: models of the human CD4+ T-cell immune response, Immunol Rev, 172, 335-43.
Stemmer, C., Quesnel, A., Prevost-Blondel, A., Zimmermann, C., Muller, S., Briand, J. P. and Pircher, H. (1999). Protection against lymphocytic choriomeningitis virus infection induced by a reduced peptide bond analogue of the H-2 Db-restricted CD8(+) T cell epitope GP33, J Biol Chem, 274, 5550-6.
Strauss, G., Vignali, D. A., Sehonrich, G. and Hammerling, G. J. (1994). Negative and positive selection by HLA-DR3(DRw17) molecules in transgenic mice, Immunogenetics, 40, 104-8.
Taneja, V. and David, C. S. (1999). HLA class II transgenic mice as models of human diseases, Immunol Rev, 169, 67-79.
Tsuji, M., Bergmann, C. C., Takita-Sonoda, Y., Murata, K., Rodrigues, E. G., Nussenzweig, R. S. and Zavala, F. (1998). Recombinant Sindbis viruses expressing a cytotoxic T-lymphocyte epitope of a malaria parasite or of influenza virus elicit protection against the corresponding pathogen in mice, J Virol, 72, 6907-10.
Vandenbark, A. A., Rich, C., Mooney, J., Zamora, A., Wang, C., Huan, J., Fugger, L., Offner, H., Jones, R. and Burrows, G. G. (2003). Recombinant TCR ligand induces tolerance to myelin oligodendrocyte glycoprotein 35-55 peptide and reverses clinical and histological signs of chronic experimental autoimmune encephalomyelitis in HLA-DR2 transgenic mice, J Immunol, 171, 127-33.
Wang, Y., Lobigs, M., Lee, E., Koskinen, A. and Mullbacher, A. (2006). CD8(+) T cell-mediated immune responses in West Nile virus (Sarafend strain) encephalitis are independent of gamma interferon, J Gen Virol, 87, 3599-609.
Wheeler, D. L., Barrett, T., Benson, D. A., Bryant, S. H., Canese, K., Church, D. M., DiCuccio, M., Edgar, R., Federhen, S., Helmberg, W., Kenton, D. L., Khovayko, O., Lipman, D. J., Madden, T. L., Maglott, D. R., Ostell, J., Pontius, J. U., Pruitt, K. D., Schuler, G. D., Schriml, L. M., Sequeira, E., Sherry, S. T., Sirotkin, K., Starchenko, G., Suzek, T. O., Tatusov, R., Tatusova, T. A., Wagner, L. and Yaschenko, E. (2005). Database resources of the National Center for Biotechnology Information, Nucleic Acids Res, 33, D39-45.

We claim:

1. A polypeptide comprising: one or more discontinuous segments of one or more proteins of a Flavivirus, said segments comprising at least 9 contiguous amino acid residues selected from SEQ ID NO: 1-206.

2. The polypeptide of claim 1 which further comprises: (a) a LAMP-1 lumenal sequence comprising SEQ ID NO: 207, and (b) a LAMP transmembrane and cytoplasmic tail comprising SEQ ID NO: 208, wherein the lumenal sequence is amino-terminal to the one or more discontinuous segments of the proteins of Flavivirus which are amino-terminal to the LAMP transmembrane and cytoplasmic tail.

3. The polypeptide of claim 1 wherein the segments are from a single Flavivirus.

4. The polypeptide of claim 1 wherein the segments are from a plurality of flaviviruses.

5. The polypeptide of claim 3 wherein the segments are from Yellow Fever Virus.

6. The polypeptide of claim 3 wherein the segments are from Dengue Virus.

7. The polypeptide of claim 3 wherein the segments are from West Nile Virus.

8. The polypeptide of claim 3 wherein the segments are from Japanese encephalitis virus.

9. A polynucleotide encoding the polypeptide of claim 1 or 2.

10. The polynucleotide of claim 9 wherein codons encoding the polypeptide are optimized according to most frequent human codon usage.

11. The polynucleotide of claim 9 comprising SEQ ID NO: 209 encoding the LAMP-1 lumenal sequence and SEQ ID NO: 210 encoding the transmembrane and cytoplasmic tail of LAMP-1.

12. A nucleic acid vector which comprises the polynucleotide of claim 9.

13. The nucleic acid vector of claim 12 which is a DNA virus.

14. The nucleic acid vector of claim 12 which is a RNA virus.

15. The nucleic acid vector of claim 12 which is a plasmid.

16. A host cell which comprises a nucleic acid vector of claim 12.

17. A method of producing a polypeptide comprising, culturing a host cell according to claim 16 under conditions in which the host cell expresses the polypeptide.

18. The method of claim 17 further comprising, harvesting the peptide from the culture medium or host cells.

19. A method of producing a cellular vaccine comprising:
transfecting antigen presenting cells with a nucleic acid vector according to claim 11, whereby the antigen presenting cells express the polypeptide.

20. The method of claim 19 wherein the antigen presenting cells are dendritic cells.

21. A method of making a vaccine, comprising: mixing together the polypeptide of claim 1 and an immune adjuvant.

22. The method of claim 21 wherein the adjuvant is selected from the group consisting of alum, lecithin, squalene, and a Toll-like receptors (TLRs) adaptor molecule.

23. A vaccine composition comprising the polypeptide of claim 1 or 2.

24. A method of immunizing a human or other animal subject, comprising:
administering to the human or other animal subject a polypeptide of claim 1 or a nucleic acid vector according to claim 12 or a host cell according to claim 16, in an amount effective to elicit Flavivirus-specific T cell activation.

25. The method of claim 24 further comprising administering to the subject a live or attenuated Flavivirus vaccine.

26. The method of claim 24 further comprising administering an immune adjuvant to the subject.

27. The method of claim 24 wherein the administration is oral, mucosal, nasal, intramuscular, intravenous, intradermal, intranasal, subcutaneous, or via electroporation.

28. A method of identifying a Flavivirus, comprising:
hybridizing a polynucleotide according to claim 9 or its complement to a Flavivirus genome, wherein hybridization of the genome to the polynucleotide indicates a species of the Flavivirus.

29. The method of claim 28 wherein the polynucleotide is from 15-90 nucleotides in length.

30. A method of identifying a Flavivirus, comprising:
contacting proteins from a virus-infected cell with an antibody which specifically binds to a polypeptide of claim 1, wherein specific binding to the proteins indicates a species of Flavivirus.

31. A method of identifying a Flavivirus, comprising:
contacting a polypeptide of claim 1 with a blood sample from a patient, wherein binding of the polypeptide to an antibody in the blood sample or T cells in the blood sample indicates a species of Flavivirus.


SEQUENCE LISTING (347 sequences)

SEQ ID NO: 1 - protein, 10 aa, Flavirus
VLGSQEGAMH

SEQ ID NO: 2 - protein, 11 aa, Flavirus
VHTWTEQYKFQ

SEQ ID NO: 3 - protein, 12 aa, Flavirus
AVHADMGYWIES

SEQ ID NO: 4 - protein, 11 aa, Flavirus
HTLWSNGVLES

SEQ ID NO: 5 - protein, 9 aa, Flavirus
GPWHLGKLE

SEQ ID NO: 6 - protein, 9 aa, Flavirus
GLYGNGVVT

SEQ ID NO: 7 - protein, 9 aa, Flavirus
LTIMDLHPG

SEQ ID NO: 8 - protein, 11 aa, Flavirus
LMRRGDLPVWL

SEQ ID NO: 9 - protein, 9 aa, Flavirus
QRTPQDNQL

SEQ ID NO: 10 - protein, 13 aa, Flavirus
PASAWTLYAVATT

SEQ ID NO: 11 - protein, 20 aa, Flavirus
HYAIIGPGLQAKATREAQKR

SEQ ID NO: 12 - protein, 13 aa, Flavirus
AAGIMKNPTVDGI

SEQ ID NO: 13 - protein, 9 aa, Flavirus
FWNTTIAVS

SEQ ID NO: 14 - protein, 9 aa, Flavirus
SGVEGEGLH

SEQ ID NO: 15 - protein, 11 aa, Flavirus
IFKLTYQNKVV

SEQ ID NO: 16 - protein, 20 aa, Flavirus
DQRGSGQVGTYGLNTFTNME

SEQ ID NO: 17 - protein, 11 aa, Flavirus
PTSRTTWSIHA

SEQ ID NO: 18 - protein, 11 aa, Flavirus
SVAMKHLTSFK

SEQ ID NO: 19 - protein, 14 aa, Flavirus
GGITYTDLARYVVL

SEQ ID NO: 20 - protein, 9 aa, Flavirus
LDTYRIILL

SEQ ID NO: 21 - protein, 9 aa, Flavirus
MAKKKGAVL

SEQ ID NO: 22 - protein, 15 aa, Flavirus
GLALTSTGWFSPTTI

SEQ ID NO: 23 - protein, 15 aa, Flavirus
AAITGSSRRLDVKLD

SEQ ID NO: 24 - protein, 10 aa, Flavirus
GKLTPYWGSV

SEQ ID NO: 25 - protein, 14 aa, Flavirus
YGGPWRFDRKWNGT

SEQ ID NO: 26 - protein, 11 aa, Flavirus
DVQVIVVEPGK

SEQ ID NO: 27 - protein, 9 aa, Flavirus
SYVSAIVQG

SEQ ID NO: 28 - protein, 20 aa, Flavirus
LRGLPVRYQTSAVQREHQGN

SEQ ID NO: 29 - protein, 10 aa, Flavirus
ASAAQRRGRV

SEQ ID NO: 30 - protein, 11 aa, Flavirus
LDNIHMPNGLV

SEQ ID NO: 31 - protein, 10 aa, Flavirus
QLYGPEREKA

SEQ ID NO: 32 - protein, 11 aa, Flavirus
LATFFLWAAEV

SEQ ID NO: 33 - protein, 9 aa, Flavirus
GTKIAGTLL

SEQ ID NO: 34 - protein, 12 aa, Flavirus
ALLLMVVLIPEP

SEQ ID NO: 35 - protein, 9 aa, Flavirus
AANEYGMLE

SEQ ID NO: 36 - protein, 14 aa, Flavirus
SQAGSLFVLPRGVP

SEQ ID NO: 37 - protein, 10 aa, Flavirus
TDLDLTVGLV

SEQ ID NO: 38 - protein, 12 aa, Flavirus
ERENHLRGECHT

SEQ ID NO: 39 - protein, 10 aa, Flavirus
LARAIIELTY

SEQ ID NO: 40 - protein, 9 aa, Flavirus
EIVMKDGRS

SEQ ID NO: 41 - protein, 9 aa, Flavirus
RARISPGAG

SEQ ID NO: 42 - protein, 12 aa, Flavirus
MSGRKAQGKTLG

SEQ ID NO: 43 - protein, 14 aa, Flavirus
WCPDSMEYNCPNLS

SEQ ID NO: 44 - protein, 9 aa, Flavirus
GRMGERQLQ

SEQ ID NO: 45 - protein, 10 aa, Flavirus
ALLVLAVGPA

SEQ ID NO: 46 - protein, 17 aa, Flavirus
KCVTVMAPDKPSLDISL

SEQ ID NO: 47 - protein, 10 aa, Flavirus
DLTLPWQSGS

SEQ ID NO: 48 - protein, 9 aa, Flavirus
HLVEFEPPH

SEQ ID NO: 49 - protein, 12 aa, Flavirus
VLIEVNPPFGDS

SEQ ID NO: 50 - protein, 16 aa, Flavirus
GDSRLTYQWHKEGSSI

SEQ ID NO: 51 - protein, 10 aa, Flavirus
RNMTMSMSMI

SEQ ID NO: 52 - protein, 9 aa, Flavirus
KRELKCGDG

SEQ ID NO: 53 - protein, 15 aa, Flavirus
KYSYYPEDPVKLASI

SEQ ID NO: 54 - protein, 13 aa, Flavirus
EEGKCGLNSVDSL

SEQ ID NO: 55 - protein, 14 aa, Flavirus
HEMWRSRADEINAI

SEQ ID NO: 56 - protein, 24 aa, Flavirus
YQRGTHPFSRIRDGLQYGWKTWGK

SEQ ID NO: 57 - protein, 29 aa, Flavirus
FSPGRKNGSFIIDGKSRKECPFSNRVWNS

SEQ ID NO: 58 - protein, 30 aa, Flavirus
ILGAAVNGKKSAHGSPTFWMGSHEVNGTWM

SEQ ID NO: 59 - protein, 9 aa, Flavirus
LDYKECEWP

SEQ ID NO: 60 - protein, 10 aa, Flavirus
THTIGTSVEE

SEQ ID NO: 61 - protein, 15 aa, Flavirus
MFMPRSIGGPVSSHN

SEQ ID NO: 62 - protein, 19 aa, Flavirus
IPGYKVQTNGPWMQVPLEV

SEQ ID NO: 63 - protein, 10 aa, Flavirus
CTMPPVSFHG

SEQ ID NO: 64 - protein, 11 aa, Flavirus
DGCWYPMEIRP

SEQ ID NO: 65 - protein, 13 aa, Flavirus
HAVPFGLVSMMIA

SEQ ID NO: 66 - protein, 11 aa, Flavirus
LLGAMLVGQVT

SEQ ID NO: 67 - protein, 13 aa, Flavirus
NNGGDAMYMALIA

SEQ ID NO: 68 - protein, 15 aa, Flavirus
GFGLRTLWSPRERLV

SEQ ID NO: 69 - protein, 10 aa, Flavirus
KDTSMQKTIP

SEQ ID NO: 70 - protein, 11 aa, Flavirus
GLTQPFLGLCA

SEQ ID NO: 71 - protein, 10 aa, Flavirus
SIPVNEALAA

SEQ ID NO: 72 - protein, 9 aa, Flavirus
GVLAGLAFQ

SEQ ID NO: 73 - protein, 12 aa, Flavirus
MENFLGPIAVGG

SEQ ID NO: 74 - protein, 9 aa, Flavirus
LMMLVSVAG

SEQ ID NO: 75 - protein, 11 aa, Flavirus
WEEEAEISGSS

SEQ ID NO: 76 - protein, 10 aa, Flavirus
EQGEFKLLSE

SEQ ID NO: 77 - protein, 11 aa, Flavirus
VMTSLALVGAA

SEQ ID NO: 78 - protein, 12 aa, Flavirus
SGDVLWDIPTPK

SEQ ID NO: 79 - protein, 12 aa, Flavirus
GIFQSTFLGASQ

SEQ ID NO: 80 - protein, 21 aa, Flavirus
TFLGASQRGVGVAQGGVFHTM

SEQ ID NO: 81 - protein, 19 aa, Flavirus
PSWASVKEDLVAYGGSWKL

SEQ ID NO: 82 - protein, 12 aa, Flavirus
WDGEEEVQLIAA

SEQ ID NO: 83 - protein, 9 aa, Flavirus
VVNVQTKPS

SEQ ID NO: 84 - protein, 10 aa, Flavirus
NGGEIGAVAL

SEQ ID NO: 85 - protein, 20 aa, Flavirus
YGNGILVGDNSFVSAISQTE

SEQ ID NO: 86 - protein, 20 aa, Flavirus
PGAGKTRRFLPQILAECARR

SEQ ID NO: 87 - protein, 11 aa, Flavirus
AECARRRLRTL

SEQ ID NO: 88 - protein, 9 aa, Flavirus
RRRLRTLVL

SEQ ID NO: 89 - protein, 17 aa, Flavirus
GLDVKFHTQAFSAHGSG

SEQ ID NO: 90 - protein, 15 aa, Flavirus
MCHATLTYRMLEPTR

SEQ ID NO: 91 - protein, 12 aa, Flavirus
AHFLDPASIAAR

SEQ ID NO: 92 - protein, 24 aa, Flavirus
ARGWAAHRARANESATILMTATPP

SEQ ID NO: 93 - protein, 29 aa, Flavirus
ATPPGTSDEFPHSNGEIEDVQTDIPSEPW

SEQ ID NO: 94 - protein, 14 aa, Flavirus
WILADKRPTAWFLP

SEQ ID NO: 95 - protein, 9 aa, Flavirus
AWFLPSIRA

SEQ ID NO: 96 - protein, 19 aa, Flavirus
LPSIRAANVMAASLRKAGK

SEQ ID NO: 97 - protein, 10 aa, Flavirus
IAEMGANLCV

SEQ ID NO: 98 - protein, 12 aa, Flavirus
KVAIKGPLRISA

SEQ ID NO: 99 - protein, 22 aa, Flavirus
GRIGRNPNRDGDSYYYSEPTSE

SEQ ID NO: 100 - protein, 16 aa, Flavirus
NAHHVCWLEASMLLDN

SEQ ID NO: 101 - protein, 18 aa, Flavirus
MLLDNMEVRGGMVAPLYG

SEQ ID NO: 102 - protein, 16 aa, Flavirus
KTPVSPGEMRLRDDQR

SEQ ID NO: 103 - protein, 11 aa, Flavirus
GLKTNDRKWCF

SEQ ID NO: 104 - protein, 17 aa, Flavirus
LRPRWCDERVSSDQSAL

SEQ ID NO: 105 - protein, 15 aa, Flavirus
RAYRNALSMMPEAMT

SEQ ID NO: 106 - protein, 9 aa, Flavirus
AAWTVYVGI

SEQ ID NO: 107 - protein, 10 aa, Flavirus
LSPMLHHWIK

SEQ ID NO: 108 - protein, 18 aa, Flavirus
MLHHWIKVEYGNLSLSGI

SEQ ID NO: 109 - protein, 12 aa, Flavirus
QSASVLSFMDKG

SEQ ID NO: 110 - protein, 9 aa, Flavirus
PFMKMNISV

SEQ ID NO: 111 - protein, 13 aa, Flavirus
LILPGIKAQQSKL

SEQ ID NO: 112 - protein, 9 aa, Flavirus
DIEEAPEMP

SEQ ID NO: 113 - protein, 10 aa, Flavirus
LYEKKLALYL

SEQ ID NO: 114 - protein, 13 aa, Flavirus
KKLALYLLLALSL

SEQ ID NO: 115 - protein, 11 aa, Flavirus
SVAMCRTPFSL

SEQ ID NO: 116 - protein, 26 aa, Flavirus
PLIEGNTSLLWNGPMAVSMTGVMRGN

SEQ ID NO: 117 - protein, 16 aa, Flavirus
GKTLGEVWKRELNLLD

SEQ ID NO: 118 - protein, 10 aa, Flavirus
ARRHLAEGKV

SEQ ID NO: 119 - protein, 9 aa, Flavirus
HLAEGKVDT

SEQ ID NO: 120 - protein, 14 aa, Flavirus
AEGKVDTGVAVSRG

SEQ ID NO: 121 - protein, 9 aa, Flavirus
VAVSRGTAK

SEQ ID NO: 122 - protein, 20 aa, Flavirus
GTAKLRWFHERGYVKLEGRV

SEQ ID NO: 123 - protein, 10 aa, Flavirus
WCYYAAAQKE

SEQ ID NO: 124 - protein, 13 aa, Flavirus
YAAAQKEVSGVKG

SEQ ID NO: 125 - protein, 12 aa, Flavirus
EKWLACGVDNFC

SEQ ID NO: 126 - protein, 11 aa, Flavirus
EKLELLQRRFG

SEQ ID NO: 127 - protein, 9 aa, Flavirus
LLQRRFGGT

SEQ ID NO: 128 - protein, 23 aa, Flavirus
TFTVNQTSRLLMRRMRRPTGKVT

SEQ ID NO: 129 - protein, 15 aa, Flavirus
LPIGTRSVETDKGPL

SEQ ID NO: 130 - protein, 15 aa, Flavirus
DNDNPYRTWHYCGSY

SEQ ID NO: 131 - protein, 11 aa, Flavirus
IEEVTRMAMTD

SEQ ID NO: 132 - protein, 9 aa, Flavirus
RMAMTDTTP

SEQ ID NO: 133 - protein, 19 aa, Flavirus
KEKVDTRAKDPPAGTRKIM

SEQ ID NO: 134 - protein, 10 aa, Flavirus
VVNRWLFRHL

SEQ ID NO: 135 - protein, 16 aa, Flavirus
QWKTANEAVQDPKFWE

SEQ ID NO: 136 - protein, 10 aa, Flavirus
KLSEFGKAKG

SEQ ID NO: 137 - protein, 13 aa, Flavirus
EDHWASRENSGGG

SEQ ID NO: 138 - protein, 9 aa, Flavirus
GLQYLGYVI

SEQ ID NO: 139 - protein, 10 aa, Flavirus
EADLDDEQEI

SEQ ID NO: 140 - protein, 11 aa, Flavirus
MEMTYKNKVVK

SEQ ID NO: 141 - protein, 10 aa, Flavirus
YKNKVVKVLR

SEQ ID NO: 142 - protein, 13 aa, Flavirus
YALNTITNLKVQL

SEQ ID NO: 143 - protein, 10 aa, Flavirus
ALSHLNAMSK

SEQ ID NO: 144 - protein, 15 aa, Flavirus
MSKVRKDISEWQPSK

SEQ ID NO: 145 - protein, 11 aa, Flavirus
GRGRVSPGNGW

SEQ ID NO: 146 - protein, 10 aa, Flavirus
ACLSKAYANM

SEQ ID NO: 147 - protein, 22 aa, Flavirus
SKAYANMWSLMYFHKRDMRLLS

SEQ ID NO: 148 - protein, 10 aa, Flavirus
KRQDKLCGSL

SEQ ID NO: 149 - protein, 18 aa, Flavirus
LVAAVIGWMLGSNTMQRV

SEQ ID NO: 150 - protein, 16 aa, Flavirus
LATVSDLSTKAACPTM

SEQ ID NO: 151 - protein, 18 aa, Flavirus
FACSTKAIGRTILKENIK

SEQ ID NO: 152 - protein, 18 aa, Flavirus
GIDTNAYYVMTVGTKTFL

SEQ ID NO: 153 - protein, 16 aa, Flavirus
TFLVHREWFMDLNLPW

SEQ ID NO: 154 - protein, 18 aa, Flavirus
VTVNPFVSVATANAKVLI

SEQ ID NO: 155 - protein, 16 aa, Flavirus
IVVGRGEQQINHHWHK

SEQ ID NO: 156 - protein, 16 aa, Flavirus
LAVGGVLLFLSVNVHA

SEQ ID NO: 157 - protein, 19 aa, Flavirus
DTGCAIDISRQELRCGSGV

SEQ ID NO: 158 - protein, 11 aa, Flavirus
RSVSRLEHQMW

SEQ ID NO: 159 - protein, 18 aa, Flavirus
GLTSTRMFLKVRESNTTE

SEQ ID NO: 160 - protein, 18 aa, Flavirus
THTLWGDGILESDLIIPV

SEQ ID NO: 161 - protein, 17 aa, Flavirus
VFGGITYTDVLRYVILV

SEQ ID NO: 162 - protein, 15 aa, Flavirus
TDVLRYVILVGAAFA

SEQ ID NO: 163 - protein, 18 aa, Flavirus
MATFKIQPVFMVASFLKA

SEQ ID NO: 164 - protein, 18 aa, Flavirus
EIPDVLNSLAVAWMILRA

SEQ ID NO: 165 - protein, 19 aa, Flavirus
LAVAWMILRAITFTTTSNV

SEQ ID NO: 166 - protein, 18 aa, Flavirus
ALLTPGLRCLNLDVYRIL

SEQ ID NO: 167 - protein, 16 aa, Flavirus
CLNLDVYRILLLMVGI

SEQ ID NO: 168 - protein, 19 aa, Flavirus
GLFNPMILAAGLIACDPNR

SEQ ID NO: 169 - protein, 10 aa, Flavirus
GWPATEVMTA

SEQ ID NO: 170 - protein, 9 aa, Flavirus
PMTIAGLMF

SEQ ID NO: 171 - protein, 17 aa, Flavirus
DISWESDAEITGSSERV

SEQ ID NO: 172 - protein, 18 aa, Flavirus
NFQLMNDPGAPWKIWMLR

SEQ ID NO: 173 - protein, 18 aa, Flavirus
ISAYTPWAILPSVVGFWI

SEQ ID NO: 174 - protein, 17 aa, Flavirus
ILPSVVGFWITLQYTKR

SEQ ID NO: 175 - protein, 17 aa, Flavirus
VYRIMTRGLLGSYQAGA

SEQ ID NO: 176 - protein, 23 aa, Flavirus
WHTTKGAALMSGEGRLDPYWGSV

SEQ ID NO: 177 - protein, 18 aa, Flavirus
GSVKEDRLCYGGPWKLQH

SEQ ID NO: 178 - protein, 18 aa, Flavirus
CYGGPWKLQHKWNGQDEV

SEQ ID NO: 179 - protein, 16 aa, Flavirus
LQHKWNGQDEVQMIVV

SEQ ID NO: 180 - protein, 18 aa, Flavirus
IVVEPGKNVKNVQTKPGV

SEQ ID NO: 181 - protein, 17 aa, Flavirus
YISAIVQGERMDEPIPA

SEQ ID NO: 182 - protein, 17 aa, Flavirus
PAGFEPEMLRKKQITVL

SEQ ID NO: 183 - protein, 18 aa, Flavirus
IRYQTSAVPREHNGNEIV

SEQ ID NO: 184 - protein, 23 aa, Flavirus
DPFPESNSPISDLQTEIPDRAWN

SEQ ID NO: 185 - protein, 11 aa, Flavirus
RVIDSRKSVKP

SEQ ID NO: 186 - protein, 16 aa, Flavirus
CYGGHTNEDDSNFAHW

SEQ ID NO: 187 - protein, 17 aa, Flavirus
NEDDSNFAHWTEARIML

SEQ ID NO: 188 - protein, 15 aa, Flavirus
AHWTEARIMLDNINM

SEQ ID NO: 189 - protein, 18 aa, Flavirus
ARIMLDNINMPNGLIAQF

SEQ ID NO: 190 - protein, 18 aa, Flavirus
NMPNGLIAQFYQPEREKV

SEQ ID NO: 191 - protein, 17 aa, Flavirus
EVITKLGERKILRPRWI

SEQ ID NO: 192 - protein, 18 aa, Flavirus
ERKILRPRWIDARVYSDH

SEQ ID NO: 193 - protein, 17 aa, Flavirus
SQIGLIEVLGKMPEHFM

SEQ ID NO: 194 - protein, 17 aa, Flavirus
HFMGKTWEALDTMYVVA

SEQ ID NO: 195 - protein, 18 aa, Flavirus
IALIALLSVMTMGVFFLL

SEQ ID NO: 196 - protein, 18 aa, Flavirus
VMTMGVFFLLMQRKGIGK

SEQ ID NO: 197 - protein, 18 aa, Flavirus
VLGVATFFCWMAEVPGTK

SEQ ID NO: 198 - protein, 18 aa, Flavirus
PATAWSLYAVTTAVLTPL

SEQ ID NO: 199 - protein, 18 aa, Flavirus
DYINTSLTSINVQASALF

SEQ ID NO: 200 - protein, 17 aa, Flavirus
PFVDVGVSALLLAAGCW

SEQ ID NO: 201 - protein, 18 aa, Flavirus
LITAAAVTLWENGASSVW

SEQ ID NO: 202 - protein, 18 aa, Flavirus
GWLSCLSITWTLIKNMEK

SEQ ID NO: 203 - protein, 15 aa, Flavirus
TWTLIKNMEKPGLKR

SEQ ID NO: 204 - protein, 10 aa, Flavirus
GGAKGRTLGE

SEQ ID NO: 205 - protein, 9 aa, Flavirus
VEDWLHRGP

SEQ ID NO: 206 - protein, 10 aa, Flavirus
EREAHLRGEC

SEQ ID NO: 207 - protein, 380 aa, Homo sapiens
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENS

SEQ ID NO: 208 - protein, 36 aa, Homo sapiens
TLIPIAVGGALAGLVLIVLIAYLVGRKRSHAGYQTI

SEQ ID NO: 209 - DNA, 1140 bp, Homo sapiens
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagc

SEQ ID NO: 210 - DNA, 117 bp, Homo sapiens
acgctgatccccatcgctgtgggtggtgccctggcggggctggtcctcatcgtcctcatcgcctacctcgtcggcaggaagaggagtcacgcaggctaccagactatctagggtacc

SEQ ID NO: 211 - protein, 17 aa, Flavivirus
GKSRAVNMLKRGMPRVL

SEQ ID NO: 212 - protein, 17 aa, Flavivirus
STKATRYLVKTESWILR

SEQ ID NO: 213 - protein, 18 aa, Flavivirus
LVKTESWILRNPGYALVA

SEQ ID NO: 214 - protein, 17 aa, Flavivirus
LRNPGYALVAAVIGWML

SEQ ID NO: 215 - protein, 18 aa, Flavivirus
LVAAVIGWMLGSNTMQRV

SEQ ID NO: 216 - protein, 18 aa, Flavivirus
MLGSNTMQRVVFVVLLLL

SEQ ID NO: 217 - protein, 16 aa, Flavivirus
RVVFVVLLLLVAPAYS

SEQ ID NO: 218 - protein, 18 aa, Flavivirus
PTIDVKMMNMEAANLAEV

SEQ ID NO: 219 - protein, 18 aa, Flavivirus
AEVRSYCYLATVSDLSTK

SEQ ID NO: 220 - protein, 16 aa, Flavivirus
LATVSDLSTKAACPTM

SEQ ID NO: 221 - protein, 18 aa, Flavivirus
LSTKAACPTMGEAHNDKR

SEQ ID NO: 222 - protein, 15 aa, Flavivirus
RGWGNGCGLFGKGSI

SEQ ID NO: 223 - protein, 18 aa, Flavivirus
FACSTKAIGRTILKENIK

SEQ ID NO: 224 - protein, 18 aa, Flavivirus
GRTILKENIKYEVAIFVH

SEQ ID NO: 225 - protein, 18 aa, Flavivirus
IKYEVAIFVHGPTTVESH

SEQ ID NO: 226 - protein, 18 aa, Flavivirus
PAAPSYTLKLGEYGEVTV

SEQ ID NO: 227 - protein, 18 aa, Flavivirus
GIDTNAYYVMTVGTKTFL

SEQ ID NO: 228 - protein, 16 aa, Flavivirus
TFLVHREWFMDLNLPW

SEQ ID NO: 229 - protein, 18 aa, Flavivirus
TLMEFEEPHATKQSVIAL

SEQ ID NO: 230 - protein, 18 aa, Flavivirus
EKLQLKGTTYGVCSKAFK

SEQ ID NO: 231 - protein, 18 aa, Flavivirus
VTVNPFVSVATANAKVLI

SEQ ID NO: 232 - protein, 17 aa, Flavivirus
KVLIELEPPFGDSYIVV

SEQ ID NO: 233 - protein, 16 aa, Flavivirus
IVVGRGEQQINHHWHK

SEQ ID NO: 234 - protein, 18 aa, Flavivirus
EQQINHHWHKSGSSIGKA

SEQ ID NO: 235 - protein, 18 aa, Flavivirus
HKSGSSIGKAFTTTLKGA

SEQ ID NO: 236 - protein, 18 aa, Flavivirus
WDFGSVGGVFTSVGKAVH

SEQ ID NO: 237 - protein, 18 aa, Flavivirus
VFTSVGKAVHQVFGGAFR

SEQ ID NO: 238 - protein, 18 aa, Flavivirus
VHQVFGGAFRSLFGGMSW

SEQ ID NO: 239 - protein, 18 aa, Flavivirus
FRSLFGGMSWITQGLLGA

SEQ ID NO: 240 - protein, 17 aa, Flavivirus
SWITQGLLGALLLWMGI

SEQ ID NO: 241 - protein, 18 aa, Flavivirus
LGALLLWMGINARDRSIA

SEQ ID NO: 242 - protein, 16 aa, Flavivirus
LAVGGVLLFLSVNVHA

SEQ ID NO: 243 - protein, 19 aa, Flavivirus
DTGCAIDISRQELRCGSGV

SEQ ID NO: 244 - protein, 18 aa, Flavivirus
RQELRCGSGVFIHNDVEA

SEQ ID NO: 245 - protein, 18 aa, Flavivirus
GVFIHNDVEAWMDRYKYY

SEQ ID NO: 246 - protein, 18 aa, Flavivirus
GLTSTRMFLKVRESNTTE

SEQ ID NO: 247 - protein, 17 aa, Flavivirus
RLNDTWKLERAVLGEVK

SEQ ID NO: 248 - protein, 18 aa, Flavivirus
THTLWGDGILESDLIIPV

SEQ ID NO: 249 - protein, 16 aa, Flavivirus
ILESDLIIPVTLAGPR

SEQ ID NO: 250 - protein, 18 aa, Flavivirus
EGRVEIDFDYCPGTTVTL

SEQ ID NO: 251 - protein, 18 aa, Flavivirus
GKLITDWCCRSCTLPPLR

SEQ ID NO: 252 - protein, 17 aa, Flavivirus
SGCWYGMEIRPQRHDEK

SEQ ID NO: 253 - protein, 18 aa, Flavivirus
IDPFQLGLLVVFLATQEV

SEQ ID NO: 254 - protein, 17 aa, Flavivirus
VFGGITYTDVLRYVILV

SEQ ID NO: 255 - protein, 15 aa, Flavivirus
TDVLRYVILVGAAFA

SEQ ID NO: 256 - protein, 17 aa, Flavivirus
FAESNSGGDVVHLALMA

SEQ ID NO: 257 - protein, 18 aa, Flavivirus
MATFKIQPVFMVASFLKA

SEQ ID NO: 258 - protein, 18 aa, Flavivirus
EIPDVLNSLAVAWMILRA

SEQ ID NO: 259 - protein, 19 aa, Flavivirus
LAVAWMILRAITFTTTSNV

SEQ ID NO: 260 - protein, 18 aa, Flavivirus
ALLTPGLRCLNLDVYRIL

SEQ ID NO: 261 - protein, 16 aa, Flavivirus
CLNLDVYRILLLMVGI

SEQ ID NO: 262 - protein, 19 aa, Flavivirus
GLFNPMILAAGLIACDPNR

SEQ ID NO: 263 - protein, 18 aa, Flavivirus
GWPATEVMTAVGLMFAIV

SEQ ID NO: 264 - protein, 18 aa, Flavivirus
MFAAFVISGKSTDMWIER

SEQ ID NO: 265 - protein, 17 aa, Flavivirus
DISWESDAEITGSSERV

SEQ ID NO: 266 - protein, 18 aa, Flavivirus
NFQLMNDPGAPWKIWMLR

SEQ ID NO: 267 - protein, 18 aa, Flavivirus
ISAYTPWAILPSVVGFWI

SEQ ID NO: 268 - protein, 17 aa, Flavivirus
ILPSVVGFWITLQYTKR

SEQ ID NO: 269 - protein, 15 aa, Flavivirus
GGVLWDTPSPKEYKK

SEQ ID NO: 270 - protein, 18 aa, Flavivirus
DTPSPKEYKKGDTTTGVY

SEQ ID NO: 271 - protein, 17 aa, Flavivirus
VYRIMTRGLLGSYQAGA

SEQ ID NO: 272 - protein, 18 aa, Flavivirus
GAGVMVEGVFHTLWHTTK

SEQ ID NO: 273 - protein, 15 aa, Flavivirus
VFHTLWHTTKGAALM

SEQ ID NO: 274 - protein, 16 aa, Flavivirus
WHTTKGAALMSGEGRL

SEQ ID NO: 275 - protein, 18 aa, Flavivirus
GSVKEDRLCYGGPWKLQH

SEQ ID NO: 276 - protein, 18 aa, Flavivirus
CYGGPWKLQHKWNGQDEV

SEQ ID NO: 277 - protein, 16 aa, Flavivirus
LQHKWNGQDEVQMIVV

SEQ ID NO: 278 - protein, 17 aa, Flavivirus
GQDEVQMIVVEPGKNVK

SEQ ID NO: 279 - protein, 18 aa, Flavivirus
IVVEPGKNVKNVQTKPGV

SEQ ID NO: 280 - protein, 18 aa, Flavivirus
PIVDKNGDVIGLYGNGVI

SEQ ID NO: 281 - protein, 17 aa, Flavivirus
VIGLYGNGVIMPNGSYI

SEQ ID NO: 282 - protein, 17 aa, Flavivirus
YISAIVQGERMDEPIPA

SEQ ID NO: 283 - protein, 17 aa, Flavivirus
PAGFEPEMLRKKQITVL

SEQ ID NO: 284 - protein, 18 aa, Flavivirus
MLRKKQITVLDLHPGAGK

SEQ ID NO: 285 - protein, 18 aa, Flavivirus
GKTRRILPQIIKEAINRR

SEQ ID NO: 286 - protein, 18 aa, Flavivirus
PQIIKEAINRRLRTAVLA

SEQ ID NO: 287 - protein, 17 aa, Flavivirus
NRRLRTAVLAPTRVVAA

SEQ ID NO: 288 - protein, 17 aa, Flavivirus
VLAPTRVVAAEMAEALR

SEQ ID NO: 289 - protein, 16 aa, Flavivirus
VAAEMAEALRGLPIRY

SEQ ID NO: 290 - protein, 18 aa, Flavivirus
IRYQTSAVPREHNGNEIV

SEQ ID NO: 291 - protein, 18 aa, Flavivirus
PREHNGNEIVDVMCHATL

SEQ ID NO: 292 - protein, 18 aa, Flavivirus
TLTHRLMSPHRVPNYNLF

SEQ ID NO: 293 - protein, 18 aa, Flavivirus
KVELGEAAAIFMTATPPG

SEQ ID NO: 294 - protein, 17 aa, Flavivirus
ATPPGTSDPFPESNSPI

SEQ ID NO: 295 - protein, 17 aa, Flavivirus
DPFPESNSPISDLQTEI

SEQ ID NO: 296 - protein, 17 aa, Flavivirus
LQTEIPDRAWNSGYEWI

SEQ ID NO: 297 - protein, 18 aa, Flavivirus
RAWNSGYEWITEYTGKTV

SEQ ID NO: 298 - protein, 18 aa, Flavivirus
TVWFVPSVKMGNEIALCL

SEQ ID NO: 299 - protein, 18 aa, Flavivirus
EMGANFKASRVIDSRKSV

SEQ ID NO: 300 - protein, 15 aa, Flavivirus
AAQRRGRIGRNPSQV

SEQ ID NO: 301 - protein, 16 aa, Flavivirus
GRIGRNPSQVGDEYCY

SEQ ID NO: 302 - protein, 16 aa, Flavivirus
CYGGHTNEDDSNFAHW

SEQ ID NO: 303 - protein, 17 aa, Flavivirus
NEDDSNFAHWTEARIML

SEQ ID NO: 304 - protein, 15 aa, Flavivirus
AHWTEARIMLDNINM

SEQ ID NO: 305 - protein, 18 aa, Flavivirus
ARIMLDNINMPNGLIAQF

SEQ ID NO: 306 - protein, 18 aa, Flavivirus
NMPNGLIAQFYQPEREKV

SEQ ID NO: 307 - protein, 18 aa, Flavivirus
EKVYTMDGEYRLRGEERK

SEQ ID NO: 308 - protein, 17 aa, Flavivirus
EYRLRGEERKNFLELLR

SEQ ID NO: 309 - protein, 18 aa, Flavivirus
ERKNFLELLRTADLPVWL

SEQ ID NO: 310 - protein, 18 aa, Flavivirus
VWLAYKVAAAGVSYHDRR

SEQ ID NO: 311 - protein, 15 aa, Flavivirus
DRRWCFDGPRTNTIL

SEQ ID NO: 312 - protein, 18 aa, Flavivirus
FDGPRTNTILEDNNEVEV

SEQ ID NO: 313 - protein, 17 aa, Flavivirus
EVITKLGERKILRPRWI

SEQ ID NO: 314 - protein, 18 aa, Flavivirus
ERKILRPRWIDARVYSDH

SEQ ID NO: 315 - protein, 17 aa, Flavivirus
SQIGLIEVLGKMPEHFM

SEQ ID NO: 316 - protein, 17 aa, Flavivirus
HFMGKTWEALDTMYVVA

SEQ ID NO: 317 - protein, 18 aa, Flavivirus
IALIALLSVMTMGVFFLL

SEQ ID NO: 318 - protein, 18 aa, Flavivirus
VMTMGVFFLLMQRKGIGK

SEQ ID NO: 319 - protein, 18 aa, Flavivirus
VLGVATFFCWMAEVPGTK

SEQ ID NO: 320 - protein, 18 aa, Flavivirus
GEFLLDLRPATAWSLYAV

SEQ ID NO: 321 - protein, 18 aa, Flavivirus
PATAWSLYAVTTAVLTPL

SEQ ID NO: 322 - protein, 18 aa, Flavivirus
DYINTSLTSINVQASALF

SEQ ID NO: 323 - protein, 17 aa, Flavivirus
PFVDVGVSALLLAAGCW

SEQ ID NO: 324 - protein, 18 aa, Flavivirus
GCWGQVTLTVTVTAATLL

SEQ ID NO: 325 - protein, 18 aa, Flavivirus
VVNPSVKTVREAGILITA

SEQ ID NO: 326 - protein, 18 aa, Flavivirus
LITAAAVTLWENGASSVW

SEQ ID NO: 327 - protein, 18 aa, Flavivirus
GWLSCLSITWTLIKNMEK

SEQ ID NO: 328 - protein, 15 aa, Flavivirus
TWTLIKNMEKPGLKR

SEQ ID NO: 329 - protein, 18 aa, Flavivirus
LVQSYGWNIVTMKSGVDV

SEQ ID NO: 330 - protein, 18 aa, Flavivirus
CDIGESSSSAEVEEHRTI

SEQ ID NO: 331 - protein, 16 aa, Flavivirus
SAEVEEHRTIRVLEMV

SEQ ID NO: 332 - protein, 18 aa, Flavivirus
VKVLCPYMPKVIEKMELL

SEQ ID NO: 333 - protein, 18 aa, Flavivirus
SRNSTHEMYWVSRASGNV

SEQ ID NO: 334 - protein, 16 aa, Flavivirus
ECHTCIYNMMGKREKK

SEQ ID NO: 335 - protein, 17 aa, Flavivirus
AKGSRAIWFMWLGARFL

SEQ ID NO: 336 - protein, 18 aa, Flavivirus
WFMWLGARFLEFEALGFL

SEQ ID NO: 337 - protein, 18 aa, Flavivirus
REDQRGSGQVVTYALNTF

SEQ ID NO: 338 - protein, 18 aa, Flavivirus
GQVVTYALNTFTNLAVQL

SEQ ID NO: 339 - protein, 19 aa, Flavivirus
NTFTNLAVQLVRMMEGEGV

SEQ ID NO: 340 - protein, 18 aa, Flavivirus
GWYDWQQVPFCSNHFTEL

SEQ ID NO: 341 - protein, 18 aa, Flavivirus
DTACLAKSYAQMWLLLYF

SEQ ID NO: 342 - protein, 18 aa, Flavivirus
YAQMWLLLYFHRRDLRLM

SEQ ID NO: 343 - protein, 18 aa, Flavivirus
YFHRRDLRLMANAICSAV

SEQ ID NO: 344 - protein, 18 aa, Flavivirus
NWVPTGRTTWSIHAGGEW

SEQ ID NO: 345 - protein, 16 aa, Flavivirus
WMEDKTPVEKWSDVPY

SEQ ID NO: 346 - protein, 18 aa, Flavivirus
PYSGKREDIWCGSLIGTR

SEQ ID NO: 347 - protein, 17 aa, Flavivirus
TWAENIQVAINQVRAII